(19) United States
(12) Patent Application Publication
(10) Pub. No.: US 2023/0242679 A1
(43) Pub. Date: Aug. 3, 2023

(54) BISPECIFIC ANTIBODY THERAPIES

(71) Applicant: CytoAgents, Inc., Pittsburgh, PA (US)

(72) Inventors: Jodi CRAIGO, Cranberry Township, PA (US); Todd J. LORENZ, Orinda, CA (US); Arthur P. BERTOLINO, Holland, MI (US)

(73) Assignee: CytoAgents, Inc., Pittsburgh, PA (US)

(21) Appl. No.: 18/162,344

(22) Filed: Jan. 31, 2023

Related U.S. Application Data
(60) Provisional application No. 63/304,955, filed on Jan. 31, 2022.

(51) Int. Cl. C07K 16/46; A61P 35/00

(52) U.S. Cl. CPC C07K 16/468; A61P 35/00; C07K 2317/31

(57) ABSTRACT

Compositions, methods, and kits that can be used to treat cancer, CRS, and ICANS are described herein. For example, pharmaceutical compositions containing beraprost, a beraprost isomer, or salts thereof can be used as an adjuvant therapy with bispecific antibodies to treat cancer while reducing or eliminating undesired and dangerous CRS effects.

### CROSS-REFERENCE TO RELATED APPLICATIONS

This application claims priority to U.S. Provisional Patent Application No. 63/304,955, which was filed on Jan. 31, 2022, entitled Bispecific Antibody Therapies, the contents of which are incorporated by reference in its entirety.

### BACKGROUND

Bispecific antibodies (sometimes referred to as BsAbs) are antibodies with two binding sites directed at two different antigens. Naturally occurring antibodies usually only target a single antigen. Bispecific antibodies can “cross-link” two different antigen targets, such as one or more antigens on cancer cells, B cells, or NK cells and one or more antigens on cytotoxic T lymphocytes. As such, bispecific antibodies are the subject of recent research efforts and are the subject of multiple pending clinical trials.

Several bispecific antibodies are approved and commercially available. These include Blincyto (Blinatumomab; Amgen; Thousand Oaks, Calif., USA), Hemlibra (emicizumab-kxwh; Genentech; South San Francisco, Calif., USA), Rybrevant (amivantamab-vmjw; Janssen Pharmaceuticals; Beerse, Belgium), and Kimmtrak (tebentafusp-tebn; Immunocore; Oxfordshire, UK).

Potent T-cell activation induced by bsAbs pose the risk of complications such as cytokine release syndrome (“CRS”), and/or immune effector cell-associated neurotoxicity syndrome (ICANS). The bispecific antibodies cause the release of large amounts of cytokines into the blood. The cytokines released can also cause immune hyperactivation due to prolonged activation of cytokine signaling. These changes can result in acute systemic inflammation. CRS can manifest as high fever, low blood pressure, fatigue, headache, myalgia, nausea, capillary leakage, tachycardia, and potentially multiorgan failure for example, liver failure and kidney damage. CRS occurs in about 45-95% of patients. Immune effector cell-associated neurotoxicity syndrome (ICANS) effects can occur in more than 25% of the patients receiving bispecific antibody therapy. ICANS can manifest as headache, confusion, language disturbance, altered consciousness, seizures, cerebral edema.

Current treatment options for CRS include Tocilizumab (anti-IL6R) which is the first line treatment for CRS. However, Tocilizumab is not always efficacious and about 50-80% of patients treated with Tocilizumab require treatment with steroids such as dexamethasone (corticosteroid). Steroid treatment is known to adversely affect both and innate and adaptive immune response. Treatment with Tocilizumab and/or steroids can also increase risk of severe ICANS and rate of infection. New therapeutic strategies that prevent or reduce Tocilizumab and/or steroid use are needed.

The use of bispecific antibodies is a powerful and promising therapy, but there exists a need for new and improved treatments to reduce or eliminate the undesired and potentially fatal CRS and/or ICANS side effects.

### SUMMARY

Disclosed herein are methods and compositions to administer bispecific antibody therapies while reducing or eliminating CRS side effects.

Methods of treating cancer can generally comprise administering at least one bispecific antibody and a first pharmaceutical composition to a subject as an adjuvant therapy/combination therapy approach.

Methods of treating cytokine release syndrome (CRS), immune effector cell-associated neurotoxicity syndrome (ICANS), or both associated with bispecific antibody administration in a subject, by administering a first pharmaceutical composition are described herein.

The first pharmaceutical composition can comprise an effective amount of a prostacyclin/prostaglandin analog, such as analogs selected from the group consisting of carbaprostacyclin, beraprost, taprostene, nileprost, iloprost, cicaprost, ciprostene, treprostinil, bonsentan, uoprost, eptaloprost, or an isomer thereof, and pharmaceutically acceptable salts thereof. In some embodiments, the prostacyclin/prostaglandin analog is beraprost, a beraprost isomer, or a beraprost salt. The salt can be a pharmaceutically acceptable salt of beraprost. The beraprost can be a beraprost isomer, such as beraprost CT01681.

Kits for the treatment of cancer in a subject can generally comprise a first container containing a first pharmaceutical composition comprising at least an effective amount of beraprost, a beraprost isomer, or a pharmaceutically acceptable salt thereof, a second container containing at least one bispecific antibody; and instructions for the administration of the first pharmaceutical composition and at least one bispecific antibody to the subject.

### BRIEF DESCRIPTION OF THE DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1A shows that CT01681 does not interfere with the tumor-killing efficacy of CD19-targeting CAR T-cells in vitro (see line connected by square points) and dose-dependently lowers the levels of IL-6, a pro-inflammatory and CRS-inducing cytokine (see bar graph) when CAR T-cells and Raji cells are present at a 10:1 ratio.

FIG. 1B shows that CT01681 does not interfere with the tumor-killing efficacy of CD19-targeting CAR T-cells in vitro (see line connected by square points) and dose-dependently lowers the levels of TNF-α, a pro-inflammatory and CRS-inducing cytokine (see bar graph) when CAR T-cells and Raji cells are present at a 10:1 ratio.

FIG. 1C shows that CT01681 does not interfere with the tumor-killing efficacy of CD19-targeting CAR T-cells in vitro (see line connected by square points) and dose-dependently lowers the levels of IFN-γ, a pro-inflammatory and CRS-inducing cytokine (see bar graph) when CAR T-cells and Raji cells are present at a 10:1 ratio.

FIG. 1D shows IL-6 reduction upon treatment with increasing concentrations of CT01681, across three different concentrations of CAR-T cells to Raji cells.

FIG. 1E shows TNF-α reduction upon treatment with increasing concentrations of CT01681, across three different concentrations of CAR-T cells to Raji cells.

FIG. 1F shows IFN-γ reduction upon treatment with increasing concentrations of CT01681, across three different concentrations of CAR-T cells to Raji cells.

FIG. 1G shows the absence of an effect of CT01681 on of CAR-T tumor killing efficacy.

### DEFINITIONS

As used herein, the term “about” when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

As used herein the term “analog” refers to a compound, the presence of which results in a biological activity of a receptor that is the same as the biological activity resulting from the presence of a naturally occurring ligand for the receptor.

The terms “administer,” “administering” or “administration” as used herein refer to directly administering a compound or a composition to a subject.

As used herein, the term “effective amount” refers to an amount that results in measurable inhibition of at least one symptom or parameter of a specific disorder or pathological process. As used herein the term “therapeutically effective amount” of compositions of the application is an amount, which confers a therapeutic effect on the treated subject, at a reasonable benefit/risk ratio applicable to any medical treatment. The therapeutic effect may be objective (that is, measurable by some test or marker) or subjective (that is, subject gives an indication of or feels an effect or physician observes a change).

As used herein the term “immediate release” refers to pharmaceutical compositions that release the active ingredient within a short period of time.

As used herein the term “modified release” refers to pharmaceutical compositions that does not otherwise release the active ingredient immediately, for example it may release the active ingredient at a sustained or controlled rate over an extended period of time, or may release the active ingredient after a lag time after administration, or may be used optionally in combination with an immediate release composition. Modified release includes extended release, sustained release, and delayed release. The term “extended release” or “sustained release” as used herein is a dosage form that makes a drug available over an extended period of time after administration. The term “delayed release” as used herein is a dosage form that releases a drug at a time other than immediately upon administration.

The phrase “pharmaceutically acceptable salt(s)”, as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, and pamoate (that is, 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N, N′-dibenzylethylenediamine, chloroprocaine, choline, diethanolamine, dicyclohexylamine, ethylenediamine, N-methylglucamine, and procaine.

The term “preventing” may be taken to mean to prevent a specific disorder, disease, or condition and/or prevent the reoccurrence of a specific disorder, disease, or condition.

The term “substantially pure isomer” refers to a formulation or composition wherein among various isomers of a compound a single isomer is present at about 70%, or greater or at about 80% or greater, or at about 90% or greater, or at about 95% or greater, or at about 98% or greater, or at about 99% or greater, or said compound or composition comprise only a single isomer of the compound.

As used herein the terms “treat”, “treated”, or “treating” refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (that is, not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

The term “unit dosage form” refers to physically discrete units suitable as a unitary dosage for human subjects and other animals, each unit containing a predetermined quantity of active material calculated to produce the desired therapeutic effect, in association with a suitable pharmaceutical excipient.

The “weight percent” disclosed herein may be weight-to-weight percent or weight-to-volume percent, depending upon the composition.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a medical condition or disorder. Such administration encompasses co-administration of these therapeutic agents in a substantially simultaneous manner, such as in a single capsule, or dosage presentation, having a fixed ratio of active ingredients or in multiple, separate capsules for each active ingredient. In addition, such administration also encompasses use of each type of therapeutic agent in a sequential manner in the same patient, with delivery of the individual therapeutics separated by about 1-24 hours, about 1-7 days, or about 1 or more weeks. In either case, the treatment regimen will provide beneficial effects of the drug combination in treating the conditions or disorders described herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having about 1-3 cells refers to groups having about 1, about 2, or about 3 cells. Similarly, a group having about 1-5 cells refers to groups having about 1, about 2, about 3, about 4, or about 5 cells, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

### DETAILED DESCRIPTION

This disclosure is not limited to the particular systems, devices and methods described, as these may vary. The terminology used in the description is for the purpose of describing the particular versions or embodiments only, and is not intended to limit the scope.

Various methods and kits are described herein for performing bispecific antibody therapies with reduced or eliminated CRS effects in a subject. The methods can include administration of at least one bispecific antibody and the first pharmaceutical composition to the subject.

Cytokine release syndrome (“CRS”) and/or ICANS are common but serious side effects of bispecific antibody therapy. The bispecific antibodies can cause the release of large amounts of pro-inflammatory cytokines into the blood of the subject being treated with bispecific antibodies. This can cause high fever, low blood pressure (hypotension), fatigue, headache, myalgia, nausea, capillary leakage, tachycardia, and potentially liver failure and kidney damage. Compositions and methods to overcome CRS are needed to enhance the therapeutic value of bispecific antibody therapy. Various methods, compositions, and kits are described herein for performing bispecific antibody therapies with reduced or eliminated cytokine release syndrome (CRS) effects, immune effector cell-associated neurotoxicity syndrome (ICANS) effects, or combination thereof in a subject. The methods can include administration of bispecific antibodies and the first pharmaceutical composition to the subject.

In the present disclosure inventors demonstrate that the first pharmaceutical composition comprising beraprost, a beraprost isomer, or a pharmaceutically acceptable salt of beraprost, can reduce the levels of cytokines associated with CRS. In particular, the inventors observed a reduction in pro-inflammatory cytokines such as, TNFα, and INF-γ. Significantly, inventors found that beraprost, a beraprost isomer, or a pharmaceutically acceptable salt of beraprost did not interfere with T-cell activity, which is needed for activation of therapeutic immune response downstream of bispecific antibody therapy.

In some embodiments, administration of beraprost, a beraprost isomer, or a pharmaceutically acceptable salt thereof can mitigate the excessive release of proinflammatory cytokines, and thereby reduce or eliminate damage caused CRS and reduce or eliminate concurrent or subsequent immune effector cell-associated neurotoxicity syndrome (ICANS) effects. ICANS typically occurs after CRS, but sometimes can happen simultaneously. When occurring after CRS, it can be days later (for example, about 1, about 2, about 3, about 4, about 5 days more) and sometimes even weeks later. There is some variability among subjects, even among subjects with similar genetic backgrounds.

Current standard of care (SOC) for CRS includes pretreatment with tocilizumab, and post-treatment with corticosteroids and supportive care based on the observed CRS symptoms. Standard of care (SOC) for ICANS does not include use of tocilizumab but is a more intensive corticosteroid treatment and supportive care based on the observed ICANS symptoms. Tocilizumab is used to block IL-6 receptors, causing more IL-6 to be in circulation. The use of corticosteroids has significant limitations due to immune suppression and other deleterious downstream effects. Corticosteroids can be administered prophylactically, although this is not typical or common. Individuals given corticosteroid treatment typically must remain in the hospital for monitoring, thus extending their stay and increasing associated expense.

The use of an orally provided first pharmaceutical composition, such as those containing beraprost, a beraprost isomer, or a pharmaceutically acceptable salt thereof, would allow more patients to seek life-saving CAR T-cell and bispecific antibody treatments.

The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia.

Methods, compositions, and kits can be used in therapies with reduced or eliminated effects from CRS, immune effector cell-associated neurotoxicity syndrome (ICANS), or both. The methods, compositions, and kits can also be used in therapies with reduced or eliminated severity measurements. The methods, compositions, and kits can also be used in therapies with reduced or eliminated need for corticosteroid treatment, IL-6 receptor blocker therapeutics, and other biological treatments. Beraprost compound CTO1681 could potentially replace tocilizumab as an early treatment option.

Types of Cancers

In some embodiments, the bispecific antibody therapies are administered to a patient diagnosed with cancer, having cancer, or suspected of having cancer. The cancer can generally be any cancer suitable for treatment with bispecific antibody therapy. In some embodiments, the cancer can be a hematological malignancy. For example, the cancer can be B-cell lymphoma, aggressive, relapsed, or refractory diffuse large B cell lymphoma (DLBCL), primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma. In some examples, the cancer is B-cell lymphoma. In some embodiments, the cancer can be a solid tumor. Cancers suitable for treatment with bispecific antibody therapy include brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, and liver metastases.

Methods of Treating Cancer and Methods of Treating CRS and ICANS Associated with Bispecific Antibody Therapies

The compounds and pharmaceutical compositions described herein may be administered at therapeutically effective dosage levels to treat the recited conditions, disorders, and diseases.

The compounds and pharmaceutical compositions described herein may be administered at prophylactically effective dosage levels to mitigate or prevent the recited conditions, disorders, and diseases.

In some embodiments, beraprost, a beraprost isomer, or a pharmaceutically acceptable salt thereof, can be used to treat cancer. The method can comprise administering at least one bispecific antibody and a first pharmaceutical composition to the subject, wherein the first pharmaceutical composition comprises at least an effective amount of beraprost, a beraprost isomer, or a pharmaceutically acceptable salt thereof. The methods can further comprise administering to the subject a second pharmaceutical composition comprising at least one corticosteroid, tocilizumab, IL-6 receptor blocker, or combinations thereof. In some examples, the subject requires reduced treatment with the second pharmaceutical composition relative to a subject who does not receive the first pharmaceutical composition.

In some embodiments, beraprost, a beraprost isomer, or a pharmaceutically acceptable salt thereof, can be used to treat CRS, ICANS or both. In some embodiments, the CRS or ICANS can be associated with bispecific antibody administration.

In some embodiments, beraprost, a beraprost isomer, or a pharmaceutically acceptable salt thereof does not interfere with T cell mediated killing of target cells downstream of bispecific antibodies. Bispecific antibodies can be designed such that a first portion binds at least one antigen on T cells, while the second portion binds an antigen on another cell for example, a tumor cell, a B cell or an NK cell. In some embodiments, beraprost, a beraprost isomer, or a pharmaceutically acceptable salt thereof does not reduce a T cell-mediated killing of a cancer cell by more than about 5%. The cell killing can occur when T-cells become activated by the binding of the bispecific antigen and cause lysis of the other cell bound by the bispecific antibody for example, a tumor cell, a B cell or an NK cell. In some embodiments, beraprost, a beraprost isomer, or a pharmaceutically acceptable salt thereof does not reduce or inhibit T-cell mediated cell killing. In some embodiments, beraprost, a beraprost isomer, or a pharmaceutically acceptable salt thereof does not reduce T cell-mediated killing of a cell by more than about 1%, about 2%, about 3%, about 4% or about 5%%, about 6%, about 7%, about 8%, about 9%, about 10%, about 0.01% to about 0.1%, about 0.1% to about 1%, about 1% to about 10%, about 10% to about 20%, about 0.5% to about 5%, about 5% to about 15%, about 15% to about 25% or more. In some embodiments, beraprost, a beraprost isomer, or a pharmaceutically acceptable salt thereof does not inhibit or reduce T-cell activation or proliferation.

In some embodiments, beraprost, a beraprost isomer, or a pharmaceutically acceptable salt thereof can be used to treat grade 1 CRS, a grade 2 CRS, a grade 3 CRS or a grade 4 CRS. Treatment with beraprost, a beraprost isomer, or a pharmaceutically acceptable salt thereof can reduce the severity of CRS such that treatment can result in a higher grade CRS for example, grade 4 CRS becoming a lower grade CRS, for example, grade 1. In some embodiments, the treatment with beraprost, a beraprost isomer, or a pharmaceutically acceptable salt thereof can eliminate or prevent CRS. In some embodiments, grade 1 CRS can include fever of about 38° C. or more. In some embodiments, grade 1 CRS can include nausea, fatigue, headache, and can require hospitalization. In some embodiments, grade 2 CRS can include fever of about 38° C. or more and hypotension not requiring vasopressors. Grade 2 CRS can further include hypoxia or decrease oxygen requiring low-flow nasal cannula or blow-by oxygen, hypotension, and/or organ toxicity. In some embodiments, grade 3 can include fever of about 38° C. or more and hypotension requiring vasopressors with or without vasopressin treatment, organ toxicity. Grade 3 can further include, hypoxia requiring high-flow nasal cannula, facemask, nonrebreather mask, or Venturi mask. In some embodiments, grade 4 can include fever of about 38° C. or more and hypotension requiring multiple vasopressors (excluding vasopressin). Grade 4 can further include, hypoxia requiring positive pressure (CPAP, BiPAP, intubation, mechanical ventilation) and/or organ toxicity.

In some embodiments, beraprost, a beraprost isomer, or a pharmaceutically acceptable salt thereof can be used to treat grade 1 ICANS, a grade 2 ICANS, a grade 3 ICANS or a grade 4 ICANS. Treatment with beraprost, a beraprost isomer, or a pharmaceutically acceptable salt thereof can reduce the severity of CRS such that treatment can result in a higher grade ICANS for example, grade 4 ICANS becoming a lower grade ICANS, for example, grade 1 ICANS. In some embodiments, the treatment with beraprost, a beraprost isomer, or a pharmaceutically acceptable salt thereof can eliminate or prevent ICANS. In some embodiments, grade 1 ICANS can have an immune effector cell-associated encephalopathy (ICE) score: about 7-9. In some embodiments, grade 1 ICANS can include the following: consciousness: awakens spontaneously; seizure: none; motor findings: none; elevated ICP/cerebral edema: none. In some embodiments, grade 2 ICANS can include an ICE score of about 3-6. In some embodiments, grade 2 ICANS can include the following: consciousness: awakens to voice; seizure: none; motor findings: none; elevated ICP/cerebral edema: none. In some embodiments, grade 3 ICANS can include an ICE score of about 0-2. In some embodiments, grade 3 ICANS can include the following: consciousness: awakens only to tactile stimulus; seizure: any clinical seizure that resolves rapidly or nonconvulsive seizures on EEG that resolve with intervention; motor findings: none; elevated ICP/cerebral edema: focal/local edema on neuroimaging. In some embodiments, grade 4 ICANS can include an ICE score of about 0 (that is, patient or subject is unable to perform ICE). Grade 4 ICANS can include the following parameters: consciousness: subject is unarousable or requires vigorous or repetitive tactile stimuli to arouse, stupor or coma; seizure: life-threatening prolonged seizure (>5 min); or repetitive clinical or electrical seizures without return to baseline in between; motor findings: deep focal motor weakness such as hemiparesis or paraparesis; elevated ICP/cerebral edema: diffuse cerebral edema on neuroimaging; decerebrate or decorticate posturing; or cranial nerve VI palsy; or papilledema; or Cushing's triad.

In some embodiments, beraprost, a beraprost isomer, or a pharmaceutically acceptable salt thereof, can reduce the levels of one or more cytokines. Non-limiting examples of cytokines whose levels can be reduced, include, IL-1α, IL-1β, IL-2, IL-4, IL-6, IL-7, IL-8, IL-10, IL-12, IFN-γ, TNF-α, IP-10, MCP-1, MIP-1, RANTES, and/or GM-CSF. In some embodiments, the levels of one or more cytokines can be reduced by about 5%, about 10%, about 15%, about 20%, about 25%, about 30%, about 40%, about 45%, about 50%, about 55%, about 60%, about 65%, about 70%, about 75%, about 80%, about 85%, about 90%, about 95%, or more. In some embodiments, the levels of one or more cytokines can be reduced by about 1-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90%, about 85-95%, and/or about 90-100%.

In some methods, beraprost, a beraprost isomer, or a pharmaceutically acceptable salt thereof can be used in a combination therapy approach with other active pharmaceutical ingredients. For example, a method of treating cytokine release syndrome, ICANS, or both, associated with bispecific antibody administration in a subject can include administering one or more bispecific antibodies, a first pharmaceutical composition, and a second pharmaceutical composition to the subject, wherein: the first pharmaceutical composition comprises at least an effective amount of beraprost, a beraprost isomer, or a pharmaceutically acceptable salt thereof; and the second pharmaceutical composition comprises at least one corticosteroid, tocilizumab, 1L-6 receptor blocker, or combinations thereof.

An additional method of treating cytokine release syndrome, ICANS, or both, associated with bispecific antibody administration can include administering one or more bispecific antibodies and a first pharmaceutical composition to the subject, wherein: the first pharmaceutical composition comprises at least an effective amount of beraprost, a beraprost isomer, or a pharmaceutically acceptable salt thereof; and the subject experiences reduced ICANS effects, relative to a similar subject receiving bispecific antibody therapy but not receiving the first pharmaceutical composition. ICANS can be assessed and graded using a cognitive assessment tool called the “Immune Effector Cell-associated Encephalopathy (ICE) Assessment” tool, level of consciousness, presence and severity of seizures, motor control impairment, and presence of cerebral edema. In one embodiment, the subject does not experience CRS such as, for example, when the first pharmaceutical composition is administered concurrently with or prior to the at least one bispecific antibody.

A further method of treating cytokine release syndrome, ICANS, or both, associated with bispecific antibody administration in a subject can include administering one or more bispecific antibodies and a first pharmaceutical composition to the subject, wherein: the first pharmaceutical composition comprises at least an effective amount of beraprost, a beraprost isomer, or a pharmaceutically acceptable salt thereof; and the subject experiences reduced severity measurements relative to a similar subject receiving the bispecific antibodies but not receiving the first pharmaceutical composition. Severity measurements can include event grades, event duration, event incidence, incidence of ICU or hospital stays, duration of ICU or hospital stays, onset timing, mortality, interference with antibiotics or other supportive medications, or combinations thereof. Additional severity measurements include use of supportive therapies, use of medical interventions, and use of intensive medical interventions such as intubation.

An additional method of treating cytokine release syndrome, ICANS, or both, associated with bispecific antibody administration in a subject can include administering one or more bispecific antibodies and a first pharmaceutical composition to the subject, wherein: the first pharmaceutical composition comprises at least an effective amount of beraprost, a beraprost isomer, or a pharmaceutically acceptable salt thereof; and the first pharmaceutical composition is administered once onset of CRS is detected by an increased level of one or more of cytokine MIF, IL-5, IL-17A, IL-23, IFN-γ, CXCL9/MIG, GCSF, VEGF-A, and TGF-β. Alternatively or additionally, onset of CRS can be detected by an increased level of one or more of cytokines such as, CCL2, IL-2, IL-6, IL-8, IL-10, IFN-γ, TNF-α, CXCL9, CXCL-10, VEGF, CCL3, GCSF, and GMCSF The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia.

An additional method of treating cytokine release syndrome, ICANS, or both, associated with bispecific antibody administration in a subject can include administering one or more bispecific antibodies and a first pharmaceutical composition to the subject, wherein: the first pharmaceutical composition comprises at least an effective amount of beraprost, a beraprost isomer, or a pharmaceutically acceptable salt thereof; the subject experiences CRS, and/or ICANS; and the subject requires reduced treatment with at least one corticosteroid or a second pharmaceutical composition relative to a similar subject receiving bispecific antibodies but not receiving the administered first pharmaceutical composition. In some examples, the subject does not require treatment with at least one corticosteroid or a second pharmaceutical composition.

Use of the described methods and pharmaceutical compositions can result in a reduction or elimination of CRS. Reduction can be an improvement or resolution of undesirable physiological symptoms the patient subject is experiencing, a quantifiable reduction in one or more cytokine concentration, or both. The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, or other undesired property). Reduction can be relative to the effect that would be observed with administration of the at least one bispecific antibody but without administration of the first pharmaceutical composition.

Bispecific antibodies are typically delivered by infusion in one single administration, although multiple administrations are also possible. While CRS does not occur in all patients, about 45-95% of patients receiving bispecific antibody therapy do develop CRS. CRS typically has an onset within the first week and can typically occur with a median time of onset of about 24-48 hours (and a range of about 6 hours to about 9 days) post administration of bispecific antibodies. The first pharmaceutical composition can be administered starting concurrently with the bispecific antibody (that is, no delay period), or starting after a delay period. In some examples, the first pharmaceutical composition can additionally be administered one or more times prior to administration of the bispecific antibody. The delay period can be a predetermined period of time after administration of the bispecific antibody (such as about 1 day, about 2 days, about 3 days, about 4 days, about 5 days, about 6 days, about 7 days, about 8 days, about 9 days, about 10 days, about 11 days, about 12 days, about 13 days, about 14 days, or more, or ranges between any two of these values). Example ranges of the delay period include about 3 days to about 7 days, about 2 days to 5 days, about 3 days to 5 days, about 2 days to 5 days, about 4 days to 8 days, and so on. Alternatively, the delay period can last until onset of CRS is detected.

Various timings and sequences of administration of the first pharmaceutical composition are possible. For example, the first pharmaceutical composition can be administered prior to administration of the bispecific antibody therapy, on the same day as administration of the bispecific antibody therapy, after administration of the bispecific antibody therapy, and combinations thereof. For example, the first pharmaceutical composition can be administered prior to administration of the bispecific antibody therapy, on the same day as administration of the bispecific antibody therapy, and after administration of the bispecific antibody therapy. In one specific example, the first pharmaceutical composition can be administered one day prior to administration of the bispecific antibody therapy, and then continues for at least about 14 days.

Onset of CRS can be detected by generally any method, such as detecting fever, headache, anorexia, nausea, fatigue, myalgia, hypoxia, low blood pressure, hypotension, impaired coagulation, capillary leakage, tachycardia, organ system failure and so on. For example, a simple method to detect onset of CRS is detecting fever. Alternatively, onset of CRS can be detected by monitoring increased levels of one or more cytokines such as IL-1α, IL-1β, IL-2, IL-4, IL-6, IL-7, IL-8, IL-10, IL-12, IFN-γ, TNF-α, IP-10, MCP-1, MIP-1, RANTES, and GM-CSF. In some examples, onset of CRS can be detected by monitoring increased levels of one or more cytokines such as IL-6, IFN-γ, TNF-α, and IL-10. Alternatively or additionally, onset of CRS can be detected by an increased level of one or more of cytokine CCL2, IL-2, IL-6, IL-8, IL-10, IFN-γ, TNF-α, CXCL9, CXCL-10, VEGF, CCL3, GCSF, and GMCSF. In some examples, onset of CRS can be detected by monitoring increased levels of one or more cytokines such as IL-6, IFN-γ, TNF-α, and IL-10. Alternatively, or additionally, onset of CRS can be detected by monitoring increased levels of one or more cytokines such as MIF, IL-5, IL-17A, IL-23, IFN-γ, CXCL9/MIG, GCSF, VEGF-A, and TGF-β. The onset of CRS can be detected in multiple ways. For example, onset can be detected by measuring an increase of one or more cytokines or one or more inflammatory markers. Alternatively, onset of CRS can be measured by presentation of symptoms. Common symptoms include fever, hypotension, and hypoxia. In some embodiments, the first pharmaceutical compositions can reduce the levels of one or more cytokines such as, but not limited to IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

For example, after the delay period or upon detecting onset of CRS, the first pharmaceutical composition can be administered for about 1 day, about 2 days, about 3 days, about 4 days, about 5 days, about 6 days, about 7 days, about 8 days, about 9 days, about 10 days, about 11 days, about 12 days, about 13 days, about 14 days, or longer, or ranges between any two of these values. CRS typically is resolved in about one week but has been documented to persist for 30 days or beyond. In some examples, the first pharmaceutical composition can be administered for more than about 14 days, such as about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or longer, or ranges between any two of these values. Example ranges include about 1 day to about 7 days, about 1 day to about 14 days, about 1 day to about 21 days, about 1 day to about 30 days, about 7 days to about 14 days, about 7 days to about 21 days, about 7 days to about 30 days, about 14 days to about 21 days, and about 21 days to about 30 days.

The treatments can generally be performed at any effective schedule. For example, the first pharmaceutical compositions disclosed herein may be administered once, as needed, about once daily, about twice daily, about three times a day, about four times a day, about once a week, about twice a week, about three times a week, about four times a week, about five times a week, about six times a week, about seven times a week, about every other week, about every other day, or the like for one or more dosing cycles. It will be understood that the specific dose level and frequency of dosage for any particular subject can be varied and will depend upon a variety of factors including the species, age, body weight, general health, gender and diet of the subject, the mode and time of administration, rate of excretion, drug combination, and severity of the particular condition.

Administration may be performed by generally any method. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some examples, the administering comprises oral delivery, subcutaneous, inhalation, IV, or IM. In some examples, the at least one bispecific antibody and the first pharmaceutical composition can be administered by the same method or by different methods.

Bispecific Antibodies

One or more bispecific antibodies can be administered. For example, about 1, about 2, about 3, about 4, about 5, about 6, or more different bispecific antibodies can be used.

In some embodiments, the bispecific antibodies can be T cell-redirecting antibodies (TRABs) also herein referring to as T cell redirecting bispecific T cell Engager (BiTE). In some embodiments, TRABs can be a bispecific antibody consisting of CD3-binding portion that binds to T cells and cancer antigen, B cell or NK cell-binding arms. TRABs can exert a strong toxicity against cancer cells via the activation of T cells.

The at least one bispecific antibody can generally be any bispecific antibody. Specific examples of commercially available bispecific antibodies include Blincyto (Blinatumomab; Amgen; Thousand Oaks, Calif., USA), Hemlibra (emicizumab-kxwh; Genentech; South San Francisco, Calif., USA), and Rybrevant (amivantamab-vmjw; Janssen Pharmaceuticals; Beerse, Belgium).

In some embodiments, the bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

Bispecific antibodies can have one or more mechanisms of action. Example mechanisms of action include recruitment and activation of immune cells for example, T cells, blocking of immune checkpoints for example, PD1 and PDL1, blocking of inflammatory factors, blocking of dual signal pathways, and others.

In some embodiments, the bispecific antibodies can be immune checkpoints blocking antibodies, inflammatory factors blocking antibodies, dual signaling pathway blocking antibodies, and/or bispecific antibodies that block the recruitment and activation of T-cells.

Examples of clinical bispecific antibodies that recruit and activate immune cells include MGD011 (MacroGenics), AFM11 (Affimed), Blinatumomab (Amgen), AMG562 (Amgen), A-319 (Generon), RG7828 (Roche), REGN1979 (Regeneron), RG6026 (Roche), GEN3013 (Genmab), FBTA05 (Trion), Plamotamab (Xencor), AMG330 (Amgen), AMG673 (Amgen), AMV-564 (Amphivena Therapeutics), GEM333 (GEMoaB Monoclonals), GBR1342 (Glenmark Pharmaceuticals), AMG424 (Amgen), AMG420 (Amgen), AMG701 (Amgen), JNJ-64007957 (Janssen), EM801 (Celgene), PF-06863135 (Pfizer), REGN5458 (Regeneron), TNB-383B (AbbVie), APVO436 (Aptevo Therapeutics), MGD006 (MacroGenics), Xmab14045 (Xencor), JNJ-63709178 (Janssen), MCLA-117 (Merus), RG6160 (Genentech), AMG427 (Amgen), JNJ-64407564 (Janssen), AMG111 (Amgen), RG7802 (Roche), Solitomab (Amgen), Catumaxomab (Trion), Pasotuxizumab (Bayer), HPN-424 (Harpoon), AMG160 (Amgen), MOR209 (Aptevo Therapeutics), BAY2010112 (Bayer), Ertumaxomab (Trion), GBR1302 (Glenmark Pharmaceuticals), M802 (YZYBio), RG6194 (Genentech), PF06671008 (MacroGenics), MGD007 (MacroGenics), MGD009 (MacroGenics), AMG757 (Amgen), REGN4018 (Regeneron), AMG596 (Amgen), ERY974 (Chugai), Tidutamab (Xencor), huGD2-BsAb (Y-mAbs), MGD014 (Macrogenics), AFM13 (Affimed), GTB-3550 (GT Biopharma), Teclistamab-cqyv (Janssen), Mosunetuzumab-axgb (Genentech/Roche), Blinatumomab (Amgen), Elranatamab (Pfizer), Epcoritamab (GenMab/Abbvie), Glofitamab (Roche), Talquetamab (Janssen), Odronextamab (Regeneron), and TG-1801 (TG Therapeutics).

Examples of clinical bispecific antibodies that block immune checkpoints include XmAb23104 (Xencor), AK104 (Akesobio AU), MGD019 (Macrogenics), XmAb207l7 (Xencor), MEDI5752 (AstraZeneca), MGD013 (Macrogenics), RG7769 (Roche), LY3434172 (Eli Lilly), FS 118 (F-Star), KN046 (Alphamab), and LY3415244 (Eli Lilly).

Examples of clinical bispecific antibodies that block inflammatory factors include ATN103 (Ablynx), ALX0061 (Ablynx), ALX0761 (Ablynx), ALX0141 (Ablynx), SAR156597 (Sanofi), GSK2434735 (GlaxoSmithKline), RG7990 (Genentech), AMG570 (Amgen), LY3090106 (Eli), and MEDI7352 (Medimmune).

Examples of clinical bispecific antibodies that block dual signal pathways include ABT165 (AbbVie), ABL-001 (ABL Bio), Navicixizumab (Celgene/Oncomed), RG7221 (Roche), BI836880 (Ablynx), R05520985 (Roche), JNJ-61186372 (Janssen R&D), EMBO1 (Epimab Biotherapeutics), MCLA-158 (Merus), MCLA-128 (Merus), KN026 (Alphamab), MBS301 (Beijing Mabworks Biotech), ZW25 (Zymeworks), MP0274 (Molecular Partners AG), RG7386 (Roche), MGD010 (MacroGenics), RG7992 (Genentech), MEDI3902 (Medimmune), BI1034020 (Boehringer Ingelheim), and Emicizumab (Roche).

While aspects of the technology are described in terms of bispecific antibodies, additional examples of the technology can include the use of multi-specific antibodies. Multi-specific antibodies can include bispecific, trispecific, tetraspecific, and so on, as opposed to traditional monospecific antibodies.

In some embodiments, administration of beraprost, a beraprost isomer, or a pharmaceutically acceptable salt thereof can reduce or eliminate hospitalization associated with the development of CRS, ICANS or a combination thereof. In some embodiments, methods of administering beraprost, a beraprost isomer, or a pharmaceutically acceptable salt thereof can transform bispecific antibody therapy from primarily an in-patient to outpatient treatment.

Pharmaceutical Compositions

Pharmaceutical compositions described herein can be a first pharmaceutical composition or a second pharmaceutical composition. The first pharmaceutical composition can comprise at least one prostacyclin/prostaglandin analog. The second pharmaceutical composition can comprise at least one corticosteroid, tocilizumab, IL-6 receptor blocker, or combinations thereof.

Examples of prostacyclin or prostaglandin analogs include carbaprostacyclin, beraprost, taprostene, nileprost, iloprost, cicaprost, ciprostene, treprostinil, bonsentan, uoprost, eptaloprost, or an isomer thereof, and/or a pharmaceutically acceptable salt thereof. In some embodiments, the first pharmaceutical composition comprises an effective amount of beraprost, a beraprost isomer, or a pharmaceutically acceptable salt thereof. Beraprost has a chemical formula C₂₄H₃₀O₅ and has a single carboxylic acid group. In some embodiments, the prostacyclin analog is a beraprost salt such as beraprost sodium (C₂₄H₂₉NaO₅; 2,3,3a,8b-tetrahydro-2-hydroxyl-1-(3-hydroxyl-4-methyl-1-octen-6-ynyl)-1H-cyclopenta[b]benzofuran-5-butanoic acid, sodium salt). Beraprost sodium (BPS; GP1001) is a mixture of four isomers—two diastereomers (BPS-314 and BPS-315) and their two enantiomers each which are BPS-314d (CT01681; GP1681, (+)-[1R, 2R, 3aS, 8bS]-2,3,3a,8b-tetrahydro-2-hydroxy-1-[(E)-(3S,4S)-3-hydroxy-4-methyl-1-octen-6-ynyl)-1Hcyclopenta[b]benzofuran-5-butanoic acid, monosodium salt; also called esuberaprost sodium salt) and BPS-3141 (GP1684), and BPS-315d (GP1683) and BPS-3151 (GP1682). Beraprost isomers are further described in U.S. Patent Publication No. US 2014-0275237 A1. In some examples, the first pharmaceutical composition can contain 1, 2, 3, or all 4 isomers of beraprost. In some embodiments, the beraprost isomer is BPS-314d (CT01681; esuberaprost sodium salt).

Beraprost and methods for its preparation are shown in U.S. Pat. No. 7,345,181 and PCT Publication No. WO 2004/026224, entitled “Process for preparing prostaglandin derivatives and starting materials for the same”. Beraprost is commercially available from Yonsung Fine Chemicals (Gyeonggi-do, Republic of Korea). Beraprost, a beraprost isomer, or a pharmaceutically acceptable salt thereof can be present in the first pharmaceutical composition at generally any effective amount or effective concentration. Different pharmaceutical forms may have different amounts or concentrations of beraprost, a beraprost isomer, or pharmaceutically acceptable salt thereof.

In some embodiments, the first pharmaceutical composition can contain 1, 2, 3, or all 4 isomers of beraprost. Beraprost isomer refers to a beraprost molecule that has identical molecular formula to another beraprost molecule but has a distinct arrangement of their atoms in space. In some embodiments, an isomer of beraprost can be a structural isomer or a stereoisomer. In some embodiments, a structural isomer can comprise isomers in which bonds between the atoms differ but has the same molecular formula. In some embodiments, the isomer can be a stereoisomer of beraprost, wherein the bonds between the atoms are the same but the relative positions of the atoms differ. In some embodiments, a stereoisomer of beraprost can be a diastereomer of beraprost or an enantiomer of beraprost. The isomers can be different stereoisomers resulting from one or more chiral centers in their chemical structure. The different isomers of beraprost can have different biological activity, and sometimes can have no activity or even undesirable activity as compared to other desired isomers.

In some embodiments, the first pharmaceutical composition can comprise BPS-314d (CT01681; GP1681, (+)-[1R, 2R, 3aS, 8bS]-2,3,3a,8b-tetrahydro-2-hydroxy-1-[(E)-(3S,4S)-3-hydroxy-4-methyl-1-octen-6-ynyl)-1Hcyclopenta[b]benzofuran-5-butanoic acid, monosodium salt; also called esuberaprost sodium salt). In some embodiments, the first pharmaceutical composition can comprise BPS-3141 (GP1684). In some embodiments, the first pharmaceutical composition can comprise BPS-315d (GP1683). In some embodiments, the first pharmaceutical composition can comprise BPS-3151 (GP1682).

In some embodiments, the first pharmaceutical composition can include one or more enantiomers of beraprost. A common purity measurement is “enantiomeric excess” or “ee”. A racemic mixture has an ee of about 0%, while a completely pure enantiomer will have an ee value of about 100%. It is desirable an ee value of the first pharmaceutical composition of at least about 80%, at least about 85%, at least about 90%, at least about 91%, at least about 92%, at least about 93%, at least about 94%, at least about 95%, at least about 96%, at least about 97%, at least about 98%, at least about 99%, and ideally 100%.

As an example, a first pharmaceutical composition comprising beraprost isomer BPS-314d (CTO1681; GP1681; esuberaprost sodium salt) can have an ee value of at least about 80%, at least about 85%, at least about 90%, at least about 91%, at least about 92%, at least about 93%, at least about 94%, at least about 95%, at least about 96%, at least about 97%, at least about 98%, at least about 99%, and ideally 100%.

In some embodiments, one or more isomers of beraprost can be separated from the others such that only select isomers are included in the first pharmaceutical composition. In one embodiment, BPS-314d (CT01681) can be separated from the other isomers in beraprost. Separation of the isomers from beraprost can be achieved using commercially-available chiral columns. Additional purification steps can be performed.

In some embodiments, synthetic methods can be used to prepare a desired isomer (for example, enantiomer or stereoisomer) in an enhanced concentration relative to undesired enantiomers or stereoisomers.

In some embodiments, the first pharmaceutical composition can have a high purity both at the time of manufacture as well as at later times such as at time of use. In some embodiments, the first pharmaceutical composition can have a low or no level of degradation products of beraprost, a beraprost isomer, or a pharmaceutically acceptable salt thereof.

Impurities are undesired in the first pharmaceutical composition and can come from different sources. An impurity can be a component of the raw materials, residual solvents, or synthesis that was incompletely removed or purified from the final desired product. A contaminant can be a substance that is unintentionally included with the final desired product due to the manufacturing environment or other sources. Impurities and contaminants can be harmful or harmless and can be identified or unidentified. In some examples, the first pharmaceutical composition contains not more than about 0.1 wt. % impurity or contaminant, not more than about 0.2 wt. % impurity or contaminant, or not more than about 0.5 wt. % impurity or contaminant. In an ideal example, the composition does not contain detectable impurities or contaminants.

One or more degradation products can arise from various paths such as instability, degradation, or oxidation of the first pharmaceutical composition itself, or by incompatibility or reaction of the first pharmaceutical composition with another component of the composition (such as one or more excipients), moisture, or the composition packaging. In some examples, the first pharmaceutical composition contains not more than about 0.1 wt. % degradation product, not more than about 0.2 wt. % degradation product or not more than about 0.5 wt. % degradation product. In one example, the composition does not contain detectable degradation products.

The daily dose (mass) of prostacyclin/prostaglandin analog or beraprost, a beraprost isomer, or pharmaceutically acceptable salt thereof can generally be any effective amount or dosage. For example, the therapeutically effective amount (in micrograms) may include about 0.1 μg to about 100 μg, about 10 μg to about 90 μg, or about 15 μg to about 90 μg, about 0.1 μg to about 5000 μg. The mass values are the combined salt weight, that is the anion and cation together. Specific examples of therapeutically effective amounts include about 0.1 μg, about 1 μg, about 5 μg about 10 μg, about 15 μg, about 20 μg, about 30 μg, about 40 μg, about g about 50 μg, about 60 μg, about 70 μg, about 80 μg, about 90 μg, about 100 μg, and ranges between any two of these values. When administered in two or more daily doses, the amount in each dose can be added together to yield a total daily dose. For example, CTO1681 may be administered at a dose of about 15-90 μg/day divided into 3 doses, and each individual dose of about 5-30 μg.

In some embodiments, the effective amount of beraprost, a beraprost isomer, or a pharmaceutically acceptable salt thereof are present in a unit dose (mass) of the first pharmaceutical composition is at least about 1 microgram, about 1 microgram to about 100 micrograms, about 1 microgram to about 80 micrograms, about 1 microgram to about 60 micrograms, about 1 microgram to about 50 micrograms, about 1 microgram to about 40 micrograms, about 51 microgram to about 30 micrograms, about 1 microgram to about 20 micrograms, about 1 mg to about 10 micrograms, or about 1 microgram to about 5 micrograms, or any value between these ranges. Specific examples include about 1 microgram, about 5 micrograms, about 10 micrograms, about 25 micrograms, about 50 micrograms, about 75 micrograms, about 100 micrograms, about 60 micrograms to about 360 micrograms, or ranges between any two of these values.

In some embodiments, the amount of beraprost, a beraprost isomer, or a pharmaceutically acceptable salt thereof can be calculated based on the presence of a single desired isomer. For example, if a single isomer, such as BPS-314d (CT01681; also esuberaprost sodium salt) is desired at an amount of about 15 micrograms to about 90 micrograms, this is equivalent to an amount of about 60 micrograms to about 360 micrograms of a racemic mixture of four isomers (where the amount of a single isomer is one-quarter of the mass).

In some embodiments, the first pharmaceutical composition comprising beraprost, a beraprost isomer, or a pharmaceutically acceptable salt thereof achieves a Cₘₐₓ of about 0.01 nanomolar to about 10 nanomolar, about 0.01 nanomolar to about 5 nanomolar, about 0.01 nanomolar to about 3 nanomolar, about 0.01 nanomolar to about 2 nanomolar, about 0.01 nanomolar to about 1 nanomolar, about 0.01 nanomolar to about 0.5 nanomolar, or any values between these ranges. Specific examples include about 0.01 nanomolar, about 0.05 nanomolar, about 0.075 nanomolar, about 0.1 nanomolar, about 0.5 nanomolar, about 1 nanomolar, about 2 nanomolar, about 5 nanomolar, or about 10 nanomolar.

In some embodiments, the first pharmaceutical composition comprising beraprost, a beraprost isomer, or a pharmaceutically acceptable salt thereof achieves a Tₘₐₓ at about 0.1 hour to about 5 hours, about 0.1 hour to about 4 hours, about 0.1 hour to about 3 hours, about 0.1 hour to about 2 hours, about 0.1 hour to about 1 hours, or any specific value between these ranges. Specific examples include about 0.1 hour, about 0.5 hour, about 1 hour, about 1.5 hours, about 1.7 hours, about 2 hours, or about 5 hours.

In some embodiments, the first pharmaceutical composition comprising beraprost, a beraprost isomer, or a pharmaceutically acceptable salt thereof achieves an AUC of about 0.01 ng·hr/mL to about 30 ng·hr/mL over a 48 hour period, about 0.01 ng·hr/mL to about 20 ng·hr/mL over a 48 hour period, about 0.01 ng·hr/mL to about 10 ng·hr/mL over a 48 hour period, about 0.01 ng·hr/mL to about 5 ng·hr/mL over a 48 hour period, about 0.01 ng·hr/mL to about 3 ng·hr/mL over a 48 hour period, about 0.01 ng·hr/mL to about 2 ng·hr/mL over a 48 hour period, or about 0.01 ng·hr/mL to about 1 ng·hr/mL over a 48 hour period. Specific examples include about 0.01 ng·hr/mL, about 0.05 ng·hr/mL, about 0.1 ng·hr/mL, about 0.5 ng·hr/mL, about 1 ng·hr/mL, about 2 ng·hr/mL, about 5 ng·hr/mL, about 10 ng·hr/mL, or about 30 ng·hr/mL.

In some examples, the first pharmaceutical composition can further comprise at least one anti-inflammatory component such as at least one corticosteroid or at least one therapeutic monoclonal antibody.

In some examples, the first pharmaceutical composition further comprises one or more pharmaceutically acceptable excipients. Examples of pharmaceutically acceptable excipients that may be present in the composition include but not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some examples, the pharmaceutic composition can further comprise one or more exemplary fillers. Examples of exemplary fillers include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some examples, the first pharmaceutical composition further comprises one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof. Disintegrants may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the first pharmaceutical composition further comprises one or more binders, including but not limited to celluloses such as hydroxypropylcellulose, methyl cellulose, and hydroxypropylmethylcellulose; starches such as corn starch, pregelatinized starch, and hydroxpropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some examples, the first pharmaceutical composition further comprises one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition, or any value between these ranges.

In some examples, the first pharmaceutical composition further comprises one or more lubricants, including but not limited to stearic acid, magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some examples, the first pharmaceutical composition further comprises one or more glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the first pharmaceutical composition is a tablet and further comprises a top coat, such as hydroxypropyl-methylcellulose coating or polyvinyl alcohol coating, and are available under the trade name Opadry, such as Opadry White, Opadry II (Opadry is a registered trademark of BPSI Holdings LLC, Wilmington, Del., USA). Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the first pharmaceutical composition can further comprise one or more preservative agents. Examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the first pharmaceutical composition can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some examples, the first pharmaceutical composition can further comprise one or more antioxidants. Examples of antioxidants include flavonoids, anthocyanidins, anthocyanins, proanthocyanidins, or combinations thereof. Antioxidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

Physical Form of the Pharmaceutical Composition

The first pharmaceutical compositions can generally be in any physical form suitable for use in treating a subject. These forms can be referred to as a unit dosage form, such as an individual pill or tablet. In some examples, the first pharmaceutical composition can be formulated as tablets, capsules, granules, powders, liquids, suspensions, gels, syrups, slurries, suppositories, patches, nasal sprays, aerosols, injectables, implantable sustained-release formulations, or mucoadherent films. In some examples, the first pharmaceutical composition may be formed as a tablet, a bi-layer tablet, a capsule, a multiparticulate, a drug coated sphere, a matrix tablet, or a multicore tablet. A physical form can be selected according to the desired method of treatment. In some examples, the physical form can be a liquid, for example for oral or IV, IP, IM, or IT administration.

The first pharmaceutical compositions can be manufactured by various conventional methods such as conventional mixing, dissolving, granulating, dragee-making, levigating, emulsifying, encapsulating, entrapping, or lyophilizing processes. The first pharmaceutical compositions can be formulated in a conventional manner using one or more physiologically acceptable carriers, diluents, excipients, or auxiliaries that facilitate processing of the active agent into preparations that can be used pharmaceutically. Proper formulation can be selected upon the route of administration chosen.

For topical administration the first pharmaceutical compositions described herein may be formulated as solutions, gels, ointments, creams, suspensions, and the like as are well-known in the art. Systemic compositions include, but are not limited to, those designed for administration by injection, for example, subcutaneous, intravenous injection (IV), intramuscular injection (IM), intrathecal injection (IT), intraperitoneal injection (IP), as well as those designed for transdermal, subcutaneous, transmucosal oral, or pulmonary administration. For injection, the first pharmaceutical compositions can be formulated in aqueous solutions, preferably in physiologically compatible buffers such as Hank's solution, Ringer's solution, or physiological saline buffer and/or in certain emulsion formulations. The solution can contain one or more formulatory agents such as suspending, stabilizing and/or dispersing agents. In certain examples the first pharmaceutical composition can be provided in powder form for constitution with a suitable vehicle, for example, sterile pyrogen-free water, before use. For transmucosal administration, one or more penetrants appropriate to the barrier to be permeated can be used in the formulation. Such penetrants are generally known in the art.

For oral administration, the first pharmaceutical composition can combine beraprost, a beraprost isomer, or pharmaceutically acceptable salt thereof with one or more pharmaceutically acceptable carriers well known in the art. Such carriers facilitate formulation as tablets, pills, dragees, capsules, liquids, gels, syrups, slurries, suspensions, and the like, for oral ingestion by a patient to be treated. For oral solid formulations such as, for example, powders, capsules and tablets, suitable excipients include fillers such as sugars, such as lactose, sucrose, mannitol, and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients, or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the first pharmaceutical composition can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

In some examples, the first pharmaceutical composition is an immediate release pharmaceutical composition, modified release pharmaceutical composition, or a combination thereof. In some examples, the immediate release first pharmaceutical composition releases beraprost, a beraprost isomer, or pharmaceutically acceptable salt thereof within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some embodiments, the first pharmaceutical composition is an immediate release first pharmaceutical composition comprising microcrystalline cellulose, hydroxypropyl cellulose, lactose monohydrate, pregelatinized starch, magnesium stearate, and/or purified water. In some embodiments, the composition can include a coating of prepared using Opadry® film coating process.

In some examples, the modified release composition may release beraprost, a beraprost isomer, or a pharmaceutically acceptable salt thereof at a sustained or controlled rate over an extended period of time or may release it after a lag time after administration. For example, it may be released from the composition 4 hours after administration, 8 hours after administration, 12 hours after administration, 16 hours after administration, or 24 hours after administration. Modified release compositions include extended release, sustained release, and delayed release compositions. In some examples, the modified release compositions may release about 10% in about 2 hours, about 20% in 2 hours, about 40% in about 2 hours, about 50% in about 2 hours, about 10% in about 3 hours, about 20% in 3 hours, about 40% in about 3 hours, about 50% in about 3 hours, about 10% in about 4 hours, about 20% in 4 hours, about 40% in about 4 hours, about 50% in about 4 hours, about 10% in about 6 hours, about 20% in 6 hours, about 40% in about 6 hours, or about 50% in about 6 hours.

In some examples, modified release compositions may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

The modified release compositions can also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Subjects to be Treated

The subject can generally be any mammal. Examples of subjects include a non-human primate, a human, a dog, a cat, a mouse, a rat, a cow, a goat, a sheep, a rabbit, a horse, and a pig. In some examples, the subject is a human. The terms “subject,” “individual” or “patient” are used interchangeably and as used herein are intended to include human and non-human animals. Non-human animals include all vertebrates, for example, mammals and non-mammals, such as non-human primates, sheep, dogs, rats, cats, cows, horses, chickens, amphibians, and reptiles. Examples of mammals include non-human primates, sheep, dogs, cats, cows, and horses. In some examples, the subject is a human or humans. The methods are suitable for treating humans having cancer. The subject may be symptomatic or asymptomatic.

Kits

In additional examples, kits are provided for treating cancer in a subject. The kits can comprise a first container containing a first pharmaceutical composition comprising at least an effective amount of prostacyclin/prostaglandin analog or beraprost, a beraprost isomer, or pharmaceutically acceptable salt thereof; a second container containing at least one bispecific antibody; and instructions for the administration of the first pharmaceutical composition to the subject. Any of the above-described first pharmaceutical compositions can be included in the kit. The kit can further comprise a third container, and so on containing additional pharmaceutical compositions or other active ingredients. In some examples, the kit can further comprise a third container containing at least one corticosteroid, tocilizumab, IL-6 receptor blocker, or combinations thereof. In some examples, the first container can contain a first pharmaceutical composition, a second container containing at least one bispecific antibody, a third container containing at least one corticosteroid, tocilizumab, IL-6 receptor blocker, or combinations thereof, and a fourth container can contain at least one solvent or solvents to be mixed with the first pharmaceutical composition before administering to the subject according to the instructions. In an example, the kit can comprise a first container containing beraprost, a beraprost isomer, or a pharmaceutically acceptable salt thereof, a second container containing at least one bispecific antibody, and a third container containing an aqueous solvent.

In additional examples, kits are provided for treating cytokine release syndrome, ICANS, or both, associated with bispecific antibody administration in a subject. The kits can comprise a first container containing a first pharmaceutical composition comprising at least an effective amount of prostacyclin/prostaglandin analog or beraprost, a beraprost isomer, or pharmaceutically acceptable salt thereof, a second container containing one or more bispecific antibodies; a third container containing a second pharmaceutical composition comprising at least one corticosteroid, tocilizumab, IL-6 receptor blocker, or combinations thereof; and instructions for the administration of the first pharmaceutical composition, the second pharmaceutical composition, and one or more bispecific antibodies to the subject. Any of the above-described pharmaceutical compositions can be included in the kit. The kit can further comprise a fourth container, and so on containing additional pharmaceutical compositions or other active ingredients. In some examples, the first container can contain a first pharmaceutical composition, a second container containing a second pharmaceutical composition, a third container containing one or more bispecific antibodies, and a fourth container can contain at least one solvent or solvents to be mixed with the first pharmaceutical composition before administering to the subject according to the instructions. In an example, the kit can comprise a first container containing beraprost, a beraprost isomer, or a pharmaceutically acceptable salt thereof, a second container containing a second pharmaceutical composition comprising at least one corticosteroid, tocilizumab, IL-6 receptor blocker, or combinations thereof, a third container containing one or more bispecific antibodies, and a fourth container containing an aqueous solvent.

In other examples, the kit can comprise: a first container containing a first pharmaceutical composition comprising at least an effective amount of beraprost, a beraprost isomer, or a pharmaceutically acceptable salt thereof; a second container containing a second pharmaceutical composition comprising at least one corticosteroid, tocilizumab, IL-6 receptor blocker, or combinations thereof, and instructions for the administration of the first pharmaceutical composition, the second pharmaceutical composition, and one or more bispecific antibodies to a subject. The kit can further contain a third container containing one or more bispecific antibodies. The kit can further comprise a fourth container containing water or an aqueous solution.

Provided herein is embodiment A, a method of treating cancer in a subject. The method can include administering at least one bispecific antibody and a first pharmaceutical composition to the subject. The first pharmaceutical composition can be at least an effective amount of beraprost, a beraprost isomer, or a pharmaceutically acceptable salt thereof.

In an embodiment B, the method of embodiment A can further include administering to the subject a second pharmaceutical composition that includes at least one corticosteroid, tocilizumab, IL-6 receptor blocker, or combinations thereof.

In an embodiment C, the method of embodiment A can include administering to the subject a second pharmaceutical composition that contains at least one corticosteroid, tocilizumab, IL-6 receptor blocker, or combinations thereof. The subject can require reduced treatment with the second pharmaceutical composition relative to a subject who does not receive the first pharmaceutical composition.

In an embodiment D, the method of embodiment A, the first pharmaceutical composition can reduce levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

In an embodiment E, the method of embodiment A, the first pharmaceutical composition does not reduce a T cell-mediated killing of a cancer cell by more than about 5%.

In an embodiment F, the method of embodiment A, the pharmaceutically acceptable salt is beraprost sodium.

In an embodiment G, the method of embodiment A, beraprost includes at least one of BPS-314d, BPS-3141, BPS-315d, and BPS-3151.

In an embodiment H, the method of embodiment A, the beraprost isomer is BPS-314d (esuberaprost sodium salt).

In an embodiment I, the method of embodiment A, can be for the treatment of cancer such as B-cell lymphoma, aggressive, relapsed, or refractory diffuse large B cell lymphoma, primary mediastinal B-cell lymphoma, high grade B-cell lymphoma, transformed follicular lymphoma, relapsed or refractory mantle cell lymphoma, acute lymphoblastic leukemia, Hodgkin's lymphoma, non-Hodgkin's lymphoma, acute myeloid leukemia, or multiple myeloma.

In an embodiment J, the method of embodiment A, can for the treatment of a cancer such as, brain cancer, breast cancer, glioblastoma, lung cancer, non-small-cell lung cancer, multiple myeloma, ovarian cancer, neuroblastoma, colorectal, biliary, pancreatic, mesothelioma, hepatoblastoma, embryonal sarcoma, prostate, sarcoma, or liver metastases.

In an embodiment K, the method of embodiment A, can be for the treatment of cancer is a B-cell lymphoma.

In an embodiment L, the method of embodiment A, can be for treatment of a subject such a mammal

In an embodiment M, the method of embodiment A, can be for treatment of a subject such as a non-human primate, cat, dog, pig, cow, goat, horse, sheep, or rabbit

In an embodiment N, the method of embodiment A, the subject is a human.

In an embodiment 0, the method of embodiment A, the at least one bispecific antibody and the first pharmaceutical composition can be administered to the subject concurrently.

In an embodiment P, the method of embodiment A, the at least one bispecific antibody and the first pharmaceutical composition can be administered to the subject concurrently

In an embodiment Q, the method of embodiment A, the first pharmaceutical composition can be administered to the subject after the at least one bispecific antibody is administered to the subject.

In an embodiment R, the method of embodiment A, the first pharmaceutical composition can be administered to the subject starting about 1 day, about 2 days, about 3 days, about 4 days, about 5 days, about 6 days, or about 7 days after the at least one bispecific antibody is administered to the subject.

In an embodiment 5, the method of embodiment A, the first pharmaceutical composition can be administered to the subject starting about 3 days to about 7 days after the at least one bispecific antibody is administered to the subject.

In an embodiment T, the method of embodiment A, the first pharmaceutical composition can be administered to the subject after the at least one bispecific antibody is administered to the subject; and once onset of CRS is detected.

In an embodiment U, the method of embodiment A, the first pharmaceutical composition can be administered to the subject after the at least one bispecific antibody is administered to the subject; and once onset of CRS is detected by an increased level of one or more of cytokine IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

In an embodiment V, the method of embodiment A, first pharmaceutical composition can be administered to the subject: after the at least one bispecific antibody is administered to the subject; and once onset of CRS is detected by an increased level of one or more of cytokine IL-6, IL-10, IFN-γ, and TNF-α.

In an embodiment W, the method of embodiment A, the first pharmaceutical composition can be administered to the subject:

after the at least one bispecific antibody is administered to the subject and

once onset of CRS is detected by an increased level of one or more of cytokine IL-6, IL-10, IFN-γ, and TNF-α.

In an embodiment X, the method of embodiment A, the first pharmaceutical composition can be administered for a period of about 1 day to about 30 days.

In an embodiment Y, the method of embodiment A, the first pharmaceutical composition can be administered for a period of about 7 days to about 14 days.

In an embodiment Z, the method of embodiment A, can include administering the first pharmaceutical composition to the subject before administration of the at least one bispecific antibody.

In an embodiment AA, the method of embodiment A, can include administration by oral delivery or intravenous injection (IV) delivery.

In an embodiment BB, the method of embodiment A, bispecific antibody has a mechanism of action of recruitment and activation of immune cells, blocking of immune checkpoints, blocking of inflammatory factors, blocking of dual signal pathways, or combinations thereof.

In an embodiment CC, the method of embodiment A, the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

In an embodiment DD, the method of embodiment A, the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

Provided herein is embodiment EE, a method of treating cytokine release syndrome (CRS), immune effector cell-associated neurotoxicity syndrome (ICANS), or both associated with administration of at least one bispecific antibody in a subject. The method can include administering at least one bispecific antibody to the subject and administering a first pharmaceutical composition to the subject. The first pharmaceutical composition can include at least an effective amount of beraprost, a beraprost isomer, or a pharmaceutically acceptable salt thereof.

In an embodiment FF, a method of embodiment EE, can further include administering to the subject a second pharmaceutical composition that contains at least one corticosteroid, tocilizumab, IL-6 receptor blocker, or combinations thereof.

In an embodiment GG, a method of embodiment EE, can further include administering to the subject a second pharmaceutical composition that contains at least one corticosteroid, tocilizumab, IL-6 receptor blocker, or combinations thereof. The subject can require reduced treatment with the second pharmaceutical composition relative to a subject who does not receive the first pharmaceutical composition.

In an embodiment HH, a method of embodiment EE, the first pharmaceutical composition can reduce levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

In an embodiment II, a method of embodiment EE, the CRS can be grade 1 CRS, a grade 2 CRS, a grade 3 CRS or a grade 4 CRS.

In an embodiment KK, a method of embodiment EE, the ICANS can be grade 1 ICANS, a grade 2 ICANS, a grade 3 ICANS or a grade 4 ICANS.

In an embodiment LL, a method of embodiment EE, the subject can experience reduced severity measurements associated with CRS, ICANS or both upon receiving the first pharmaceutical composition relative to subject who does not receive the first pharmaceutical composition.

In an embodiment MM, a method of embodiment EE, the pharmaceutically acceptable salt is beraprost sodium salt.

In an embodiment NN, a method of embodiment EE, the beraprost can include at least one of BPS-314d, BPS-3141, BPS-315d, and BPS-3151.

In an embodiment 00, a method of embodiment EE, the beraprost isomer is BPS-314d (esuberaprost sodium salt).

In an embodiment PP, a method of embodiment EE, the subject is a mammal.

In an embodiment QQ, a method of embodiment EE, the subject is a non-human primate, cat, dog, pig, cow, goat, horse, sheep, or rabbit.

In an embodiment RR, a method of embodiment EE, the subject is a human.

In an embodiment SS, a method of embodiment EE, the at least one bispecific antibody and the first pharmaceutical composition are administered to the subject concurrently.

In an embodiment TT, a method of embodiment EE, the first pharmaceutical composition is administered to the subject after the at least one bispecific antibody is administered to the subject.

In an embodiment UU, a method of embodiment EE, the first pharmaceutical composition is administered to the subject starting about 1 day, about 2 days, about 3 days, about 4 days, about 5 days, about 6 days, or about 7 days after the at least one bispecific antibody is administered to the subject.

In an embodiment VV, a method of embodiment EE, the first pharmaceutical composition is administered to the subject starting about 3 days to about 7 days after the at least one bispecific antibody is administered to the subject.

In an embodiment WW, a method of embodiment EE, the first pharmaceutical composition is administered to the subject after the at least one bispecific antibody is administered to the subject; and once onset of CRS is detected.

In an embodiment XX, a method of embodiment EE, the first pharmaceutical composition is administered to the subject after the at least one bispecific antibody is administered to the subject; and once onset of CRS is detected by an increased level of one or more of cytokine IL-1α, IL-1β, IL-2, IL-4, IL-6, IL-7, IL-8, IL-10, IL-12, IFN-γ, TNF-α, IP-10, MCP-1, MIP-1, RANTES, and GM-CSF.

In an embodiment YY, a method of embodiment EE, the first pharmaceutical composition is administered to the subject after the at least one bispecific antibody is administered to the subject; and once onset of CRS is detected by an increased level of one or more of cytokine IL-6, IL-10, IFN-γ, and TNF-α.

In an embodiment ZZ, a method of embodiment EE, the first pharmaceutical composition is administered to the subject after the at least one bispecific antibody is administered to the subject; and once onset of CRS is detected by an increased level of one or more of cytokine IL-6, IL-10, IFN-γ, and TNF-α.

In an embodiment AAA, a method of embodiment EE, the first pharmaceutical composition is administered to the subject after the at least one bispecific antibody is administered to the subject; and once onset of CRS is detected by an increased level of one or more of cytokine IL-6, IL-10, IFN-γ, and TNF-α.

In an embodiment BBB, a method of embodiment EE, the first pharmaceutical composition is administered for a period of about 1 day to about 30 days.

In an embodiment CCC, a method of embodiment EE, the first pharmaceutical composition is administered for a period of about 7 days to about 14 days.

In an embodiment DDD, a method of embodiment EE, administering the first pharmaceutical composition to the subject before administration of the at least one bispecific antibody.

In an embodiment EEE, a method of embodiment EE, the subject experiences reduced CRS relative to a similar subject receiving administered at least one bispecific antibody but not receiving the administered first pharmaceutical composition.

In an embodiment EEE, a method of embodiment EE, the subject does not experience CRS.

In an embodiment FFF, a method of embodiment EE, the administering includes oral delivery or intravenous injection (IV) delivery.

In an embodiment GGG, a method of embodiment EE, the at least one bispecific antibody has a mechanism of action of recruitment and activation of immune cells, blocking of immune checkpoints, blocking of inflammatory factors, blocking of dual signal pathways, or combinations thereof.

In an embodiment HHH, a method of embodiment EE, the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

In an embodiment III, a method of embodiment EE, the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody or combinations thereof.

Provided herein is embodiment JJJ, a kit for the treatment of cancer in a subject. The kit can include a first container containing a first pharmaceutical composition that includes beraprost, a beraprost isomer, or pharmaceutically acceptable salt thereof; a second container containing at least one bispecific antibody; and instructions for the administration of the first pharmaceutical composition to the subject.

In an embodiment KKK, a kit of embodiment JJJ, the beraprost can be at least one of BPS-314d, BPS-3141, BPS-315d, and BPS-3151.

In an embodiment LLL, a kit of embodiment JJJ, the beraprost isomer can be BPS-314d (esuberaprost sodium salt).

In an embodiment MMM, a kit of embodiment JJJ, can further include a third container containing at least one corticosteroid, tocilizumab, IL-6 receptor blocker, or combinations thereof.

In an embodiment NNN, a kit of embodiment JJJ, can include a fourth container containing at least one solvent.

Provided herein is embodiment 000, a kit for the treatment of cytokine release syndrome (CRS), immune effector cell-associated neurotoxicity syndrome (ICANS), or both in a subject. The kit can include a first container containing a first pharmaceutical composition that contains beraprost, a beraprost isomer, or pharmaceutically acceptable salt thereof, and a second container containing at least one bispecific antibody. The kit can also include instructions for the administration of the first pharmaceutical composition to the subject.

In an embodiment PPP, a kit of embodiment 000, the beraprost includes at least one of BPS-314d, BPS-3141, BPS-315d, and BPS-3151.

In an embodiment QQQ, a kit of embodiment 000, the beraprost isomer is BPS-314d (esuberaprost sodium salt).

In an embodiment RRR, a kit of embodiment 000, can include a third container containing at least one corticosteroid, tocilizumab, IL-6 receptor blocker, or combinations thereof.

In an embodiment SSS, a method of embodiment 000, can further include a fourth container containing at least one solvent.

### EXAMPLES

### Example 1: Evaluation of Mouse Model of CRS

This study will contain 3 groups—placebo control, positive control (tocilizumab or dexamethasone), and CTO1681 treated. The CTO1681 treated groups would have multiple arms for a dose-response determination in a murine model of CRS. These dosages will provide the information required for the secondary in vivo models of CAR-T therapy associated CRS treatment.

Humanized mice (expressing human PBMCs—HU-PBMC NSG™; commercially available from The Jackson Laboratory; Bar Harbor, Me., USA) will be given either control treatments or 1 of 5 doses of CTO1681 prior to CRS induction. To induce CRS, treated mice will be administered the antibody OKT3 intraperitoneally (IP), (anti-CD3 monoclonal antibody) to induce CRS. Mice will be sacrificed 24-48 hours post CRS-induction and cytokine production will be quantified (peripheral and in tissues).

Results will show that mice receiving CTO1681 prior to CRS induction had lower undesired cytokine production than mice in the control group. Results will also show a reduction in the rapid, acute symptoms that occur in the mouse model within the first 48 hours. Longer models would show survival benefits, but this particular Example will not be conducted to that point.

### Example 2: Mouse Model of Bispecific Antibody Therapy Induced CRS

This study will contain 3 groups-placebo control, dexamethasone control, and CT01681 dose determined from the primary CRS mouse studies of Example 1. SCID or humanized mice (expressing human PBMCs—HU-PBMC NSG™) will be injected (IP) with Raji-luc tumor cells and observed for approximately three weeks for tumor growth. Tumor burden will be assessed via bioluminescence.

CAR-T cell treatment (IP) will be performed to solicit CRS (occurs approximately 2-3 days after CAR-T infusion). Controls and CT01681 treatments (IP, BID for 7 days) will start approximately 5 hours prior to CAR-T cell transfer. At the end of the 7-day treatment regimen tumor burdens will be assessed via bioluminescence and mice sacrificed for gross histopathology as well as peripheral and tissue cytokine concentrations determined.

Results will show that mice receiving either dexamethasone or CT01681 had lower undesired cytokine production than mice in the control group, and that CTO1681 was superior to dexamethasone. While both the dexamethasone control and CT01681 will demonstrate a reduction in cytokine levels, it is expected that there will be greater survival benefits to CT01681 over dexamethasone.

### Example 3: Treatment with No Delay Period

A group of 50 human subjects having B-cell lymphoma will be divided into two groups—a control group and a CT01681 treatment group. Both groups will receive infusion of at least one bispecific antibody. The CT01681 treatment group will receive CT01681 starting with co-administration with the at least one bispecific antibody and continuing daily for seven days. Cytokines will be monitored daily for both groups. Results will show that subjects receiving CTO1681 had reduced CRS symptoms and lower undesired cytokine production than subjects in the control group.

### Example 4: Treatment with Predetermined Delay Period

A group of 80 human subjects having mantle cell lymphoma will be divided into two groups—a control group and a CT01681 treatment group. Both groups will receive infusion of at least one bispecific antibody. The CT01681 treatment group will receive CT01681 starting three days after administration with the at least one bispecific antibody and continuing daily for eleven days. Cytokines will be monitored daily for both groups. Results will show that subjects receiving CTO1681 had reduced CRS symptoms and lower undesired cytokine production than subjects in the control group.

### Example 5: Treatment with Monitored Delay Period

A group of 40 human subjects having acute myeloid leukemia will be divided into two groups—a control group and a CT01681 treatment group. Both groups will receive infusion of at least one bispecific antibody. Cytokines will be monitored daily for both groups. The CTO1681 treatment group will start to receive CTO1681 upon detection of an increase in any one of cytokines IL-6, IFN-γ, and IL-10. Treatment with CTO1681 will continue daily for ten days. Results will show that subjects receiving CT01681 had reduced CRS symptoms and lower undesired cytokine production than subjects in the control group.

### Example 6: Kit for Treatment of Cancer

A box will be configured with a first container containing an effective amount of beraprost, a beraprost isomer, or a pharmaceutically acceptable salt thereof, and written instructions for the administration of the first pharmaceutical composition and at least one bispecific antibody to a subject. The instructions can be printed on paper and placed within the box or can be a hyperlinked website having the written instructions. The box is combined with a second container containing at least one bispecific antibody. The box can optionally contain a third container containing water or an aqueous solution to dissolve the beraprost, a beraprost isomer, or a pharmaceutically acceptable salt thereof.

### Example 7: Assessment of Cytokine Release Following CT01681 Treatment

Cytokine release assays in normal human PBMC for analysis of CT01681 down regulation of cytokine production were developed and performed per the parameters evaluated in earlier pilot work. For the current assays PBMC from 5 healthy donors were procured.

The assay began with pretreatment of rested cells with either a positive control drug (dexamethasone) or CTO1681. Following pre-treatment, cells were stimulated with either LPS or Poly (I:C). Twenty-four to forty-eight hours post-stimulation supernatants were collected and tested both for viability (48 hours) as well as concentrations of 29 different cytokines (24 hours.). Viability assessments confirmed the lack of CTO1681-associated cell cytotoxicity. Statistical comparisons in individual donor results were conducted between all test groups within each donor group (between individual stimulant-treatment pairs) to determine efficacy of stimulation as well as any potential CT01681 effects on cytokine production. Additionally, statistical analysis was performed on pooled donor data for each cytokine stimulant-treatment pair to comprehensively determine cytokine suppression effects of CTO1681 for each cytokine.

Both LPS and Poly (I:C) stimulated PBMC from all five donors but there were some noted variabilities. Both stimulants failed to induce significant stimulation (stimulated cells vs. unstimulated control cells) in various cytokines from various donors. Likewise, there were donor-specific variabilities in the level of stimulation notably more often in Donor 5 who had a lower level of stimulation across all cytokines.

While these noted variations in cytokine responses were an unpredictable, unintentional result, this phenomenon does adequately represent the naturally observed variation in human immune responses. Taken together both as individual donor and pooled responses, CTO1681 treatment demonstrated significant reduction in 21 different cytokines detailed in the following Table 1, where the ** symbol indicates the 21 cytokines.

TABLE 1
Cytokine Responses

| | |
|---|---|
| LPS | Poly(I:C) |

| Cytokine | DEX | CTO1681 | DEX | CTO1681 |
|---|---|---|---|---|
| ** IL-1α | YES | YES | YES | YES |
| ** IL-1β | YES | YES | YES | YES |
| IL-2 | YES | NO | YES | NO |
| ** IL-4 | YES | YES | NO | YES |
| ** IL-5 | YES | YES | YES | YES |
| IL-6 | YES | NO | YES | NO |
| IL-7 | N/A | N/A | N/A | N/A |
| IL-8 | NO | NO | NO | NO |
| ** IL-9 | YES | YES | YES | NO |
| IL-10 | YES | NO | YES | NO |
| ** IL-12 | YES | YES | YES | YES |
| ** IL-13 | YES | YES | YES | YES |
| ** IL-15 | YES | YES | YES | YES |
| ** IL-17 | YES | YES | YES | NO |
| ** IL-18 | YES | YES | YES | YES |
| ** TNF-α | YES | YES | YES | YES |
| ** CCL2 | YES | YES | NO | YES |
| (MCP-1) | | | | |
| ** CCL3 | YES | YES | NO | NO |
| (MIP1 α) | | | | |
| ** CCL5 | YES | YES | YES | YES |
| (RANTES) | | | | |
| ** CXCL9 | YES | YES | YES | YES |
| (MIG) | | | | |
| ** CXCL10 | YES | YES | YES | YES |
| (IP-10) | | | | |
| ** IFN-γ | YES | YES | YES | YES |
| ** IFN-α2 | YES | YES | YES | YES |
| GCSF | NO | NO | NO | NO |
| ** GM-CSF | YES | YES | N/A | N/A |
| ** FGF | YES | YES | NO | NO |
| ** PDGF | YES | YES | YES | YES |
| VEGF | N/A | N/A | N/A | N/A |
| TGF-β | NO | NO | NO | NO |

These 21 include 10 cytokines not previously identified by Gemmus Pharma, Inc. studies. Further, this work indicated that IL-2, IL-6, IL-8, and IL-10 signals were not significantly reduced in this ex vivo assay. It's important to note, that these four cytokines were identified as reduced by CT01681 treatment in Gemmus Pharma's in vivo influenza (H₅N1) therapeutic studies. Moreover, IL-2, IL-6, and IL-10 were reduced by CTO1681 treatment in current CytoAgents in vivo influenza (H1N1) studies. The differences in the observed reductions in in vivo work versus ex vivo work highlights an important aspect of the inherent differences in the assays with the caveat that a negative result in ex vivo does not always equate a negative result in vivo. These results can be surprising and unexpected at times.

The final cytokine measured in these assays not analyzed in previous Gemmus Pharma, Inc. work was IFN-α. IFN-α is a type I interferon that is tightly linked to the antiviral response of the immune system which is not generally associated with suppression through suppression of NFkB induction. CT01681 had a significant reduction of the very low levels of IFN-α produced in this ex vivo system. This does not directly relate to suppression of TIFN-αL antiviral activity (IFNα was not suppressed during earlier in vivo CTO1681 influenza treatment studies). Altogether, data from Gemmus Pharma, Inc studies and the present work here confirm that the production of approximately 25 different cytokines are down regulated through treatment with CT01681.

### Example 8: Cellular Targets—Normal Human PBMC

Cytokine release assays were performed in normal human PBMC from five donors of mixed age, race, and gender. Normal human PBMC were obtained from Lonza's extensive catalog of cellular reagents. Cells were thawed according to manufacturer's instructions, washed in complete growth media, and assessed for viability using trypan blue staining. A stock cell solution of 2×10⁶ cells/mL was suspended in complete growth media for allocation into black-walled plate cell wells (2×10⁵/well) for the assay. Cells were rested at 37° C., 5% CO₂ for 1 hour.

### Example 9: Treatment, Stimulation, Cell Harvest and Cell Viability Measures

After resting the cells, 100 μl of CT01681 was added to appropriate wells at a final dosing concentration of 750 μM. Vehicle and dexamethasone (final dosing concentration of 1 M) were also added at 100 μl respectively to their appropriate wells, and all were incubated at 37° C. with 5% CO₂ for 15 minutes. For cell stimulation, 20 μL of the 1.0 ng/mL LPS solution or the 250 g/mL Poly (I:C) solution were added to appropriate wells according to the group designation. Twenty-four hours later 100 μL of supernatant were collected from all wells and stored for cytokine analysis. Approximately four hours before final harvest, AlamarBlue dye was added to all wells. At forty-eight hours post stimulation all samples were collected, and supernatants tested for cell viability. When added to cells, AlamarBlue is modified by the reducing environment of viable cells and becomes highly fluorescent. Hence, increased fluorescence after cell staining indicates increased viability. Alamar blue viability assessments demonstrated no cytotoxicity when cells were treated with CT01681 in Donors 1-5. Likewise, no signs of cytotoxicity were observed by the stimulation of cells with LPS or Poly (I:C).

### Example 10: Cytokine Readouts, End Results, and Conclusions

Cytokines were assessed on 24-hour post-stimulation collected samples on a MAGPIX instrument using Luminex multiplex technology via multiplex kits from Millipore. Concentrations of each cytokine were assessed based on a standard curve. Sample values collected by the multiplex analysis below the limit of detection were not included in final determinations. Undiluted donor IL-6 concentrations were beyond the linearity of the assay and hence the valuations were too high for the typical standard curve. Therefore IL-6 samples were re-run at a 1:5 dilution. The determined cytokine concentrations (pg/mL) results of each cytokine were plotted as individual donor stimulant-treatment pairs. Statistical comparisons on all cytokines individual donor results were conducted between control groups via a one-way ANOVA with multiple comparisons. ANOVIA analyses were between the stimulated groups (LPS only, Poly(I:C) only) and the controls (media only, dex, and vehicle) to determine efficacy of stimulation and dexamethasone positive control, as well as any potential vehicle effect. An ordinary one-way t-test was then used to compare the vehicle group to the CT01681 stimulation to determine if there was an effect with the test item. The stimulant-treatment pairs of all five donors as pooled were plotted, single means to evaluate the overall efficacy of CTO1681 cytokine suppression. Statistical analysis (Wilcoxon matched-pairs signed rank test) was performed on the pooled donor results for each cytokine plotted as well. Cytokine suppression end results were determined by evaluating both the pooled results as well as the individual donor results. CT01681 reduced 21 of the 29 cytokines analyzed in this work.

### Example 11: Observations from Ex Vivo Treatment Results

Effective stimulation of the PBMCs via LPS or Poly(I:C) was determined by comparison of the media control group to the stimulation group (non-treatment). Both mitogens successfully stimulated the majority of the cytokines, however there were variabilities both between donors and between cytokines. VEGF and TL-7 failed to stimulate consistently with either LPS or Poly(I:C) to a level above the lower limits of the standard curves and hence were not computed in the pooled analysis. The failure to stimulate is likely an artifact of the ex vivo assay which must be run with relatively short windows of observation as well as the lack of the complexities of a complete in vivo system. Additional cytokines while successfully stimulated, didn't achieve stimulation to a level of significant value as compared to unstimulated media wells. Statistically significant increases in either LPS or Poly(I:C) were detected in a minority of donors in the TGF-β, TL-2 results. Statistical significance was not achieved in the Poly(I:C) groups for GM-CSF, IL-8, IL-17A, CXCL10/IP-10, or CCL5/RANTES. Variability between the donor's general levels of stimulation was also a notable observation. Overall Donor 5 had a much lower level of stimulation across all cytokines when treated with both Poly(I:C) and LPS. Due to this lack of stimulation, Donor 5 data was not included in pooled and final analysis for IL-2, TL-4, IL-5, IL-9, IL-15, IL-12, IL-13, IL-17, IL-18, GM-CSF, and PDGF. Donor 5 stimulation for cytokines IL-10 and CXCL10/IP-10 was above the lowest limit of detection but the values were magnitudes (1-3 logs) below the other 4 donors hence while Donor 5 data was not included in the pooled analysis for these two cytokines, individual results were still considered in final determinations for CT01681 efficacy. Similarly, Donor 1 TGF-β results were all below the lower limit threshold from the standard curve so data from this donor for this cytokine were not included in the overall pooled analysis of CTO1681 efficacy. Donor 4 did have a notable lower level of overall stimulation in several cytokines (but not below the threshold for inclusion in calculations). Taken together these differences highlight the natural variation seen in generalized immune responses to mitogens within a population likely due to genetic differences between the subjects.

Much like the observations noted for the variabilities seen between groups and donors for successful stimulation, a similar unpredictability was observed with treatment group results within both stimulation methods and donors as well. CT01681 was capable of decreasing concentration in a few cytokines on which dexamethasone had no effect. Overall, CT01681 suppressed cytokine production in 21 of the 29 cytokines evaluated. Only three cytokines, GCSF, TGF-β and IL-8, experienced a lack of suppressed production with both CTO1681 and dexamethasone treatments, despite successful stimulation in both LPS and Poly(I:C) induced cells. Likewise, in some instances dexamethasone failed to suppress cytokine production while CT01681 successfully reduced produced cytokine concentrations (Poly(I:C)-stimulated cells—IL-4, CCL2/MCP1), as well as the vice versa with CT01681 failing to suppress cytokine production while dexamethasone successfully reduced the cytokine concentrations (Poly(I:C)-stimulated cells—IL-17, IL-9). Furthermore, in some instances, both dexamethasone and CTO1681 demonstrated significant reductions in cytokine production in one stimulation mitogen (LPS) but not the other (Poly(I:C)), as observed in FGF and CCL3/MIP1a. Undeniably, the complexities of assessing the study in its entirety, controls versus CTO1681 in the two different stimulants, in five donors, and 29 different cytokines, are quite formidable. Ultimately, conclusions based on evaluations of the pooled and individual donor data indicate CTO1681 suppression of cytokine production in both stimulation methods in the following cytokines—IL-1α, IL-1-β, IL-4, IL-5, IL-12, p70, IL-13, IL-15, IL-18, TNFα, CCL2/MCP-1, CCL5/RANTES, CXCL9/MIG, CXCL10/IP-10, IFN-γ, IFN-α, and PDGF; and observed suppression in cytokines from LPS stimulation alone—IL-9, IL-17, CCL3/MIP1a, GM-CSF, and FGF.

There are many operational factors to consider in the interpretation of the mechanisms and actions behind these tabulated results. Each stimulant triggers responses through different cellular receptors which in turn can then have different timing in the subsequent cellular cascade of events. Further, the window of analysis for this study—24 hours, while necessary for the nature of this ex vivo study, likely does not provide enough time for all the evaluated cytokines' production to be initiated or reach a level of significant expression. Additionally, in lacking a complete biological system, recruitment of immunological factors that can play a role in the different cytokine responses are also not present in the entirety that exists in an intact physiological system. Hence negative results should be observed with this knowledge and require the awareness that in vivo results could be somewhat different. For instance, in this ex vivo study, IL-2, IL-6, IL-8 and IL-10 were all negative for CTO1681 suppression. However, in concurrent data from in vivo lethal influenza H1N1 studies (and previous Gemmus Pharma Inc. H5N1 in vivo studies) CT01681 significantly suppressed the production of these cytokines.

CTO1681 suppression of ex vivo IFN-α activity was surprising and unexpected. IFN-α is a type 1 interferon not typically associated with the same pathways or patterns of standard proinflammatory cytokines, or more pointedly, activity through NFkB induction. Type 1 interferons are an important part of the antiviral immune response. Further, type I interferons are not typically associated with NFkB production as IFN-α is produced from different promoters and transcriptional elements than standard proinflammatory cytokines. The pathway for IFN-α, from cellular receptors to feedback loops, is entirely different from proinflammatory cytokines. Inclusion of IFN-α in this study was initially incorporated as a safety, not an efficacy measure. The results seen here are the reverse of the ex vivo/in vivo phenomenon mentioned above for IL-2, IL-6, et. al. While IFN-α suppression by CT01681 is apparent in the data in these ex vivo studies, our in vivo data indicates the contrary, or no significant repression of IFN-α concentrations by CT01681 treatment during lethal H1N1 influenza infections. Of importance when evaluating these two contrasting results is the recognition of the substantial difference in the amount of IFN-α produced in ex vivo stimulated PBMC versus active viral replication induced IFN-α levels in vivo. LPS stimulated IFNα production in PBMC was <101 pg/mL, while Poly(I.C) stimulated levels were approximately 101 pg/mL. H1N1 lethally infected mice had approximately 103 pg/mL IFN-α detected in BALF fluids. The nominal reduction in the ex vivo production of IFNα (albeit statistically significant) was less than 2-fold. At the levels of IFN-α produced during active viral replication, this small reduction would not be a significant change in BALF IFN-α. IFN-α production occurs in two phases, early and late, with the late production correlated with the high levels of IFN-α associated with viral infections. One of the transcription factors responsible for a pronounced portion of IFN-α production in the early phase is IRF7. IRF7 has an NFkB response element in its promoter. Mechanistically, this is the likely means that CTO1681 indirectly influences IFN-α production, having significant yet minimal in magnitude effects on the type 1 interferon produced as observed in this ex vivo assay.

In summary, this data and information demonstrate a broad efficacy of CT01681 cytokine suppression of 25 different cytokines identifying new cytokines (10 as compared to previous Gemmus Pharma, Inc. data) affected through ex vivo treatment of stimulated PBMC with the molecule.

### Example 12: In Vitro Analysis of CT01681 Activity in T Cell Function

The impact of CT01681 on the efficacy of CD19-targeting CAR T-cells in vitro was studied as a surrogate measure of the impact of CTO1681 on T cell function downstream of administering bispecific antibody therapy to a subject. The objective of the study was to determine if CT01681 displayed an anti-CRS phenotype while preserving anti-tumor functions of T-cells using CAR-T cells model. CD3 T cells were sorted from one healthy donor PBMCs, stimulated for a short period of time and transduced with CD19 CAR-T lentivirus (LV) containing CD28 and CD3ζ domains. CD19 CAR T-cells were expanded for 6 days and used in the assay.

CD19-targeting CD3 T-cells were treated with 5 increasing concentrations of CT01681 (0.36 nM, 1.8 nM, 9 nM, 45 nM, and 225 nM) or vehicle for 30 minutes prior to, and during co-culture with CD19+Raji lymphoma target cells. Media alone, vehicle, and positive control (dexamethasone) wells were included. Following initial treatment, CAR T-cells (Effectors) were co-cultured with fluorescently labeled CD19+Raji tumor cells (Target cells) at 3 different Effector: Target cell ratios (10:1, 5:1 and 1:1) and treated with 5 different concentrations of CT01681 or vehicle for 24 hours. Target cells (Raji cells) were fluorescently labelled with CPD (eBioscience™ Cell Proliferation Dye eFluor™) prior to co-culture to distinguish the Raji cells from the effector cells and allow for analysis of target viability by flow cytometry. Following incubation, supernatant was collected and levels of pro-inflammatory cytokines IL-6 and TNF-α were measured by multiplex Luminex assay and INF-7 was measured via time-resolved fluorescence resonance energy transfer (TR-FRET) assay. These cytokines were selected for quantification as they are early cytokines released following CAR T-cell infusion and bispecific antibody therapy administration in vivo that causes hyperactivation of the immune system resulting in acute systemic inflammation, CRS, multiorgan failure, and possible death. In addition, flow cytometry was used to measure the level of Raji tumor/target cell death following CAR T treatment across CTO1681 conditions to measure impact of CTO1681 with CAR T-cell efficacy. Co-cultures were stained with viability dye 7-AAD for measuring viability.

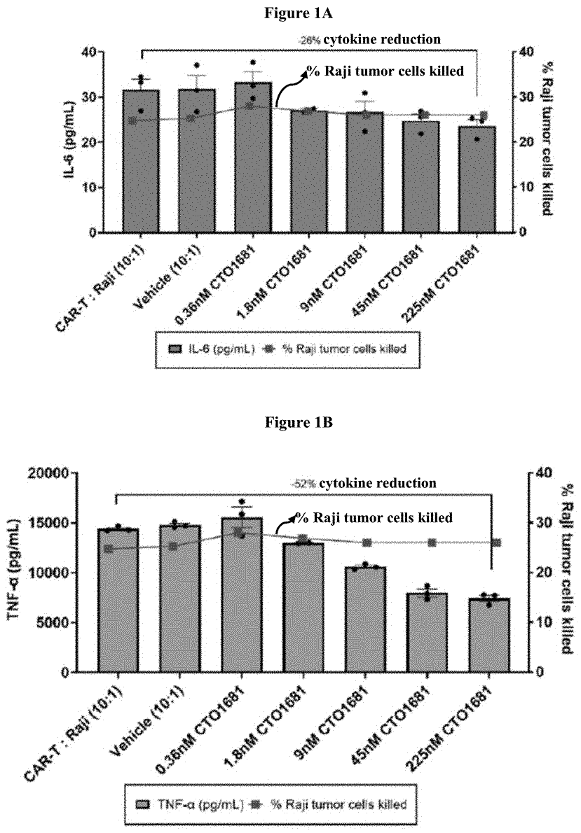

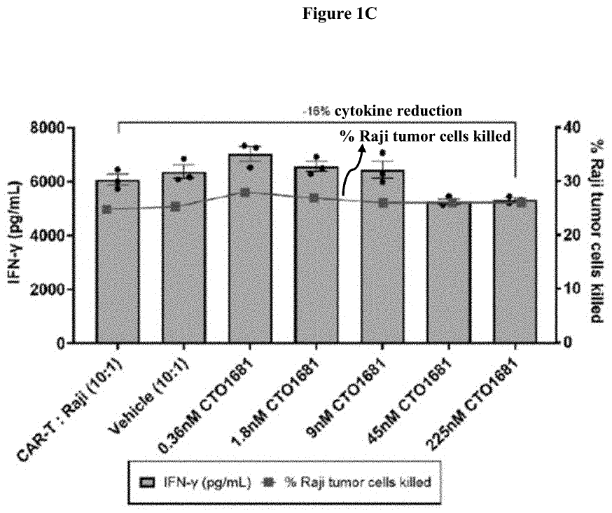

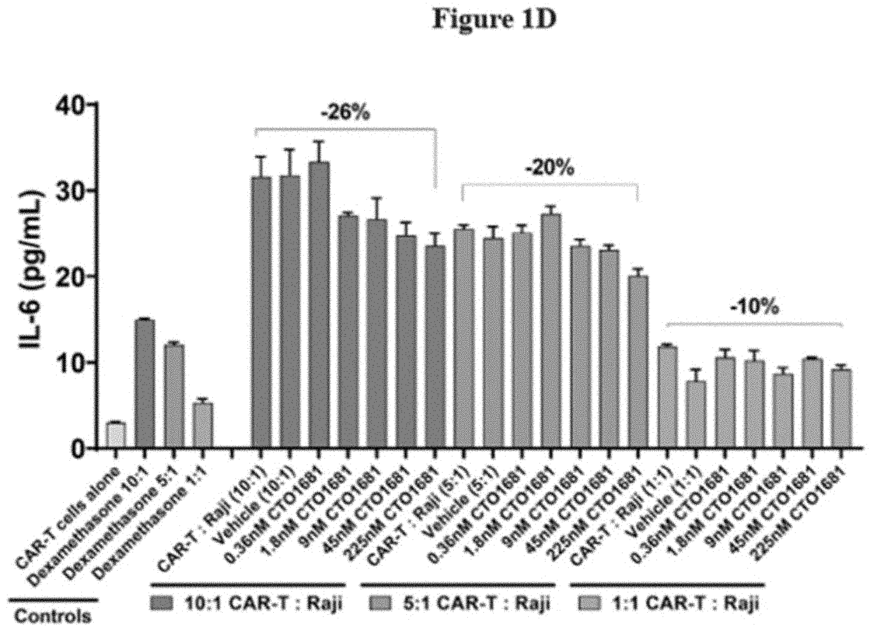

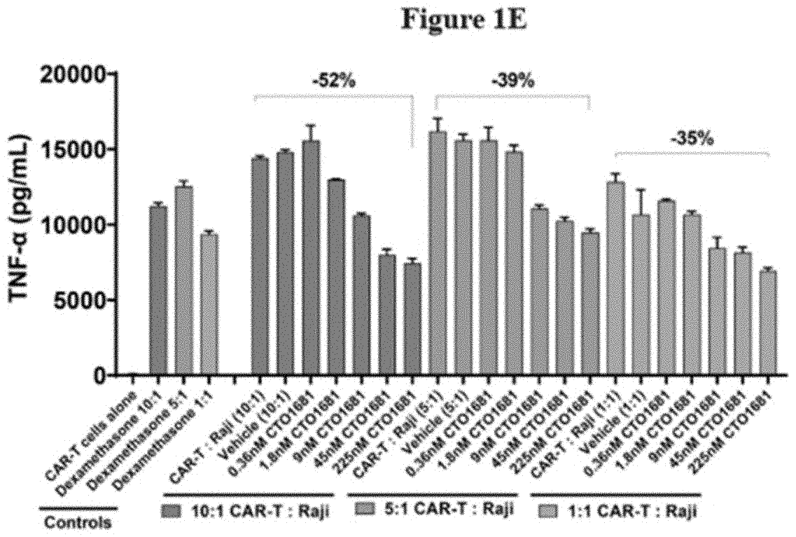

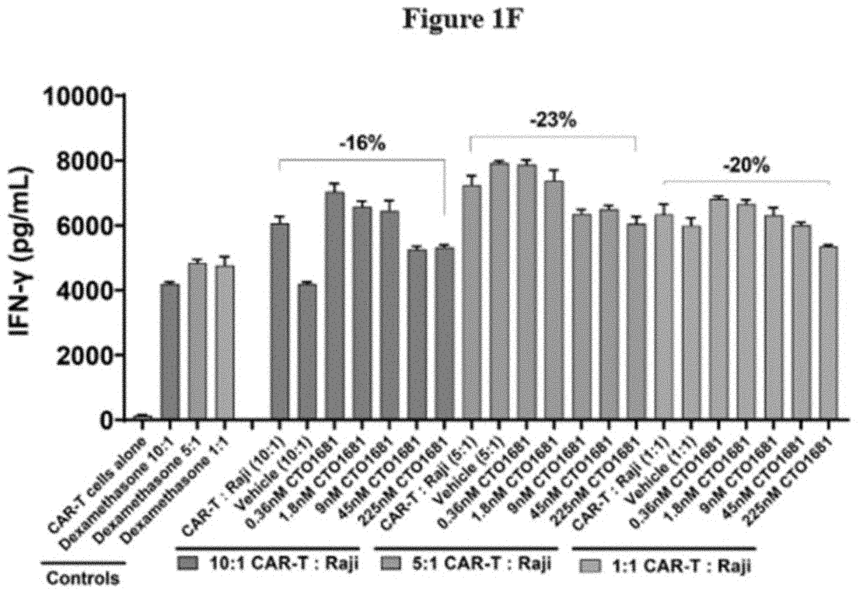

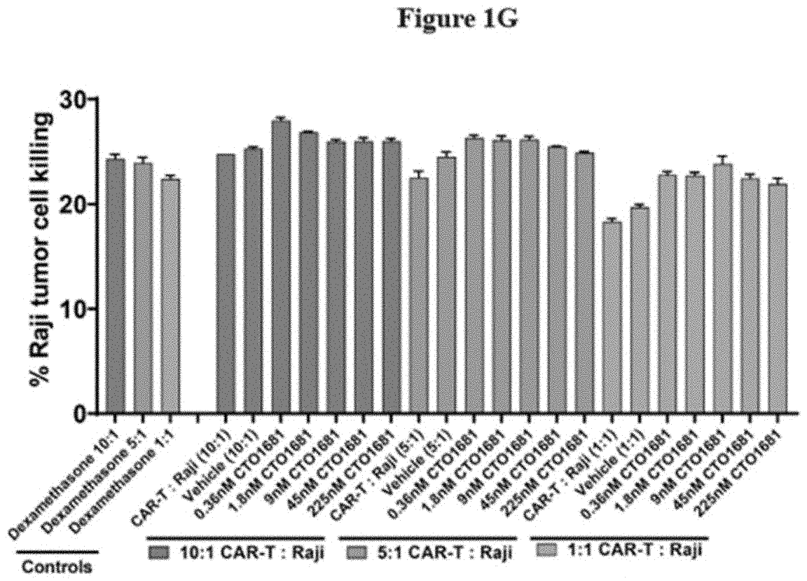

The results for 10:1 ratio of Effectors to Target cells are shown in FIG. 1A, FIG. 1B and FIG. 1C. Maximum reduction reached approximately 50% for TNF-α, 26% for IFN-γ, and 23% for IL-6, respectively. The study found that CT01681 did not impact or interfere with the tumor/target cell killing effect of CD19-directed CAR T-cells in vitro (for example, CT01681 does not interfere with CAR T-cell efficacy). All concentrations of CT01681 showed target cell killing. Based on these results, it is reasonable to conclude that T cell mediated target killing downstream of bispecific antibody administration will not impacted by administration of CTO1681.

CTO1681 also dose-dependently reduced pro-inflammatory and CRS-inducing cytokine levels IL-6, TNF-α, and INF-γ. Similar results were obtained at 5:1 and 1:1 ratios of Effectors to Target cells (FIG. 1D, FIG. 1E, FIG. 1F and FIG. 1G). Over a dozen cytokines have been shown to be elevated following CAR T-cell infusion and bispecific antibody administration in patients and CT01681 has been shown to reduce the level of each cytokine in non-CAR T models. The results here demonstrating CT01681 is able to significantly reduce the levels of IL-6, TNF-α, and IFN-γ aligns with previous findings that CT01681 can reduce each of these cytokine levels in infectious disease models in vitro and in vivo. The results also provide additional validation that the CTO1681 targets an underlying source of pro-inflammatory cytokine release from immune cells. Therefore, CT01681 is an optimal therapeutic candidate for CAR T-cell and bispecific antibody therapies. Collectively, results from this study demonstrate in vitro that CT01681 does not interfere with T-cell efficacy. It also provides strong evidence that CT01681 acts on T cells for example, CAR T-cells to significantly reduce the release of pro-inflammatory cytokines critical in initiating a hyperactive immune response and life-threatening conditions including CRS.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

What is claimed is:

1. A method of treating cancer in a subject, the method comprising administering at least one bispecific antibody and a first pharmaceutical composition to the subject, wherein the first pharmaceutical composition comprises at least an effective amount of beraprost, a beraprost isomer, or a pharmaceutically acceptable salt thereof.

2. The method of claim 1, further comprising administering to the subject a second pharmaceutical composition comprising at least one corticosteroid, tocilizumab, IL-6 receptor blocker, or combinations thereof.

3. The method of claim 1, wherein the first pharmaceutical composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

4. The method of claim 1, wherein the first pharmaceutical composition does not reduce a T cell-mediated killing of a cancer cell by more than about 5%.

5. The method of claim 1, wherein the pharmaceutically acceptable salt is beraprost sodium.

6. The method of claim 1, wherein the beraprost comprises at least one of BPS-314d, BPS-3141, BPS-315d, and BPS-3151.

7. The method of claim 1, wherein the beraprost isomer is BPS-314d (esuberaprost sodium salt).

8. The method of claim 1, wherein the at least one bispecific antibody and the first pharmaceutical composition are administered to the subject concurrently.

9. The method of claim 1, wherein the first pharmaceutical composition is administered to the subject after the at least one bispecific antibody is administered to the subject.

10. The method of claim 1, wherein the first pharmaceutical composition is administered to the subject:
after the at least one bispecific antibody is administered to the subject; and
once onset of CRS is detected.

11. The method of claim 1, wherein the first pharmaceutical composition is administered for a period of about 1 day to about 30 days.

12. The method of claim 1, further comprising administering the first pharmaceutical composition to the subject before administration of the at least one bispecific antibody.

13. The method of claim 1, wherein the administering comprises oral delivery or intravenous injection (IV) delivery.

14. The method of claim 1, wherein the at least one bispecific antibody has a mechanism of action of recruitment and activation of immune cells, blocking of immune checkpoints, blocking of inflammatory factors, blocking of dual signal pathways, or combinations thereof.

15. The method of claim 1, wherein the at least one bispecific antibody is Blincyto (Blinatumomab), Hemlibra (emicizumab-kxwh), Rybrevant (amivantamab-vmjw), Kimmtrak (tebentafusp-tebn), or combinations thereof.

16. The method of claim 1, wherein the at least one bispecific antibody is a CD19-CD3 bispecific antibody, a Factor IX-Factor X bispecific antibody, an EGFR-MET bispecific antibody, a gp100-CD3 bispecific antibody, a BCMA-CD3 bispecific antibody, a CD20-CD3 bispecific antibody, a GPRC5D-CD3 bispecific antibody, or combinations thereof.

17. A method of treating cytokine release syndrome (CRS), immune effector cell-associated neurotoxicity syndrome (ICANS), or both associated with administration of at least one bispecific antibody in a subject, the method comprising administering at least one bispecific antibody to the subject, and administering a first pharmaceutical composition to the subject; wherein:
the first pharmaceutical composition comprises at least an effective amount of beraprost, a beraprost isomer, or a pharmaceutically acceptable salt thereof.

18. The method of claim 17, further comprising administering to the subject a second pharmaceutical composition comprising at least one corticosteroid, tocilizumab, IL-6 receptor blocker, or combinations thereof.

19. The method of claim 17, wherein the first pharmaceutical composition reduces levels of one or more of cytokines of IL-1α, IL-1β, IL-2, IL-4, IL-6, IL-7, IL-8, IL-10, IL-12, IFN-γ, TNF-α, IP-10, MCP-1, MIP-1, RANTES, and GM-CSF in the subject.

20. The method of claim 17, wherein the beraprost comprises at least one of BPS-314d, BPS-3141, BPS-315d, and BPS-3151.

21. The method of claim 17, wherein the beraprost isomer is BPS-314d (esuberaprost sodium salt).

22. The method of claim 17, wherein the at least one bispecific antibody and the first pharmaceutical composition are administered to the subject concurrently.

23. The method of claim 17, wherein the first pharmaceutical composition is administered to the subject after the at least one bispecific antibody is administered to the subject.

24. The method of claim 17, wherein the first pharmaceutical composition is administered to the subject:
after the at least one bispecific antibody is administered to the subject; and
once onset of CRS is detected.

25. The method of claim 17, further comprising administering the first pharmaceutical composition to the subject before administration of the at least one bispecific antibody.

26. The method of claim 17, wherein the subject experiences reduced CRS relative to a similar subject receiving administered at least one bispecific antibody but not receiving the administered first pharmaceutical composition.

27. The method of claim 17, wherein the subject does not experience CRS.

28. A kit for the treatment of cancer in a subject, the kit comprising:
a first container containing a first pharmaceutical composition comprising beraprost, a beraprost isomer, or pharmaceutically acceptable salt thereof,
a second container containing at least one bispecific antibody; and
instructions for the administration of the first pharmaceutical composition to the subject.

29. A kit for the treatment of cytokine release syndrome (CRS), immune effector cell-associated neurotoxicity syndrome (ICANS), or both in a subject, the kit comprising:
a first container containing a first pharmaceutical composition comprising beraprost, a beraprost isomer, or pharmaceutically acceptable salt thereof,
a second container containing at least one bispecific antibody; and
instructions for the administration of the first pharmaceutical composition to the subject.
